(19) United States
(12) Patent Application Publication
(10) Pub. No.: US 2025/0298037 A1
(43) Pub. Date: Sep. 25, 2025

(54) METHODS AND COMPOSITIONS FOR ASSESSING AND TREATING PERSISTENT ACUTE KIDNEY INJURY BASED ON C-C MOTIF CHEMOKINE LIGAND 14 MEASUREMENT

(71) Applicant: Astute Medical, Inc., San Diego, CA (US)

(72) Inventors: James Patrick Kampf, San Diego, CA (US); Thomas Kwan, San Diego, CA (US); PAul McPherson, Encinitas, CA (US)

(21) Appl. No.: 18/843,553

(22) PCT Filed: Mar. 1, 2023
(86) PCT No.: PCT/US2023/063524

§ 371 Date: Sep. 3, 2024

Related U.S. Application Data
(60) Provisional application No. 63/316,377, filed on Mar. 3, 2022.
(60) Provisional application No. 63/318,338, filed on Mar. 9, 2022.
(60) Provisional application No. 63/319,206, filed on Mar. 11, 2022.

(51) Int. Cl. G01N 33/68

(52) U.S. Cl. CPC G01N 33/6893; G01N 2333/521; G01N 2800/347; G01N 2800/50; G01N 2800/52

(57) ABSTRACT

The present invention relates to methods and compositions for assessing a risk for developing persistent acute kidney injury and methods of treating a subject based on the assessment. In particular, disclosed herein are methods and compositions for detecting C—C motif chemokine ligand 14 (CCL14) for predicting the risk of persistent acute kidney injury based on changes in the level of CCL14 in two or more measurements of CCL14 in a subject and/or based on two cutoff levels of CCL14.

### CROSS REFERENCED APPLICATIONS

This application claims the benefit of U.S. Provisional Application No. 63/316,377 filed on Mar. 3, 2022, U.S. Provisional Application No. 63/318,338 filed on Mar. 9, 2022, and U.S. Provisional Application No. 63/319,206 filed on Mar. 11, 2022, each of which is herein incorporated by reference in its entirety.

### SEQUENCE LISTING

This application contains a Sequence Listing which has been submitted in electronic format and is hereby incorporated by reference in its entirety. The Sequence Listing is provided as a file entitled DN02084SeqListPCT.xml, created Mar. 1, 2023 which is 2,924-bytes in size.

### BACKGROUND

The kidney is responsible for water and solute excretion from the body. Its functions include maintenance of acid-base balance, regulation of electrolyte concentrations, control of blood volume, and regulation of blood pressure. As such, loss of kidney function through injury and/or disease results in substantial morbidity and mortality. A detailed discussion of renal injuries is provided in Harrison's Principles of Internal Medicine, 17^{th} Ed., McGraw Hill, New York, pages 1741-1830, which is herein incorporated by reference in its entirety. Renal disease and/or injury may be acute or chronic. Acute and chronic kidney disease are described as follows (from Current Medical Diagnosis & Treatment 2008, 47^{th} Ed, McGraw Hill, New York, pages 785-815, which is herein incorporated by reference in its entirety): “Acute renal failure is worsening of renal function over hours to days, resulting in the retention of nitrogenous wastes (such as urea nitrogen) and creatinine in the blood. Retention of these substances is called azotemia. Chronic renal failure (chronic kidney disease) results from an abnormal loss of renal function over months to years”.

Acute Kidney Injury (AKI, also known as acute renal failure, or ARF) is an abrupt (typically detected within about 48 hours to 1 week) reduction in glomerular filtration. AKI is a major global cause of both morbidity and mortality. It is estimated that at least half of AKI cases resolve within 72 hours. Cases of AKI that resolve within 72 hours tend to have markedly better outcomes compared to cases which persist for at least 72 hours, especially for cases of severe AKI. Oliguria lasting at least 72 hours has been identified as a criterion for initiation renal replacement therapy (RRT). See, Gaudry S, Hajage D, Schortgen F, Martin-Lefevre L, Pons B, Boulet E, et al. The New England Journal of Medicine. 2016; 375 (2): 122-33, which is herein incorporated by reference in its entirety. Recent evidence suggests that two-thirds of patients with AKI resolve their renal dysfunction within 3-7 days whereas those who persist have dramatically reduced survival over the following year. See, Kellum J A, Sileanu F E, Bihorac A, Hoste E A, Chawla L S. Am J Respir Crit Care Med. 2017;195 (6): 784-91, which is herein incorporated by reference in its entirety. Persistence of AKI at one week or more, termed acute kidney disease (AKD), is of grave importance in that it increases an individual's risk of developing chronic kidney disease and the consequences thereof. This link to chronic kidney disease (CKD) has been established over the last decade and specific recommendations for the management of patients with AKD have been proposed in order to try to influence this transition. See, Chawla L S, Bellomo R, Bihorac A, Goldstein S L, Siew E D, Bagshaw S M, et al., Nat Rev Nephrol. 2017;13 (4): 241-57; Chawla L S, Eggers P W, Star R A, Kimmel P L, The New England Journal of Medicine 2014; 371 (1): 58-66, each of which is herein incorporated by reference in its entirety. It follows that early identification of individuals at risk of AKD would enable appropriate delivery of these proposed interventions, but also may identify individuals where newer therapies to attenuate AKI could be targeted.

Not only is persistence of AKI relevant to longer term outcomes, but clinical decision-making is also critically affected by physician expectations surrounding renal recovery and the decision of when to initiate renal replacement therapy (RRT). Currently this is almost totally dependent on clinical expectations as to the likelihood of recovery with no commercially available diagnostics to aid this decision process. As such, significant controversy exists around the timing of RRT with studies showing that some patients can benefit from the earlier initiation of RRT, while other studies demonstrate that some individuals receive RRT who may not require such treatment as they will recover renal function soon. (Bagshaw S M, Lamontagne F, Joannidis M, Wald R. Critical care 2016; 20 (1): 245; Forni L G, Joannidis M. Nat Rev Nephrol 2019; 15 (1): 5-6.) It follows that early and reliable identification of those who will recover renal function may enable treatment to be stratified and avoid the incumbent risks of extracorporeal therapy.

These challenges underscore the need for better methods to detect and assess AKI, particularly in the early and subclinical stages, but also in later stages when recovery and repair of the kidney can occur. Furthermore, there is a need to better identify patients who are at risk of having persistent AKI.

### SUMMARY

Methods and compositions for evaluating renal function in a subject are provided. As described herein, measurements of C—C motif chemokine ligand 14 (CCL14) can be used for assessing the risk of persistent acute kidney injury (AKI) in a subject diagnosed as having AKI. In one aspect, a method for assessing an elevated risk for developing persistent acute kidney injury (AKI) in a subject is provided. The method comprises (a) performing an assay to detect a level of C—C motif chemokine ligand 14 (CCL14) in a urine sample obtained from the subject; and (b) determining the subject is at elevated risk for developing persistent AKI based upon the level of CCL14 detected in the urine sample being greater than a CCL14 threshold concentration of about 1.3 ng/ml.

In another aspect, a method for assessing a high risk for developing persistent acute kidney injury (AKI) in a subject is provided. The method comprising (a) performing an assay to detect a level of C—C motif chemokine ligand14 (CCL14) in a urine sample obtained from the subject; and (b) determining the subject is at high risk for developing persistent AKI based upon the level of CCL14 detected in the urine sample being greater than a CCL14 threshold concentration of about 13.0 ng/ml.

In some embodiments, the level of CCL14 detected in the urine sample is above the CCL14 threshold concentration of about 1.3 ng/ml and below a CCL14 threshold concentration of about 13.0 ng/ml.

In one embodiment, the method further comprises seeking further analysis and/or treatment from a nephrologist or specialist.

In some embodiments, the method further comprises treating the subject at elevated or high risk of having persistent AKI with one or more of renal replacement therapy (RRT), withdrawing of compounds that are known to be damaging to the kidney, performing a procedure known to be damaging to the kidney after a delay of at least about 48 hours from obtaining the sample, modifying diuretic administration, modifying dosing of renally cleared compounds, and/or administering one or more agents or measured volumes of fluid to restore normal fluid levels, electrolyte levels, or hemodynamics.

In one embodiment, the RRT comprises one or more of continuous RRT, intermittent RRT, hemodialysis, peritoneal dialysis, hemofiltration, and renal transplantation.

In one embodiment, the subject is diagnosed as having AKI.

In one embodiment, the subject has KDIGO stage 1 AKI. In one embodiment, the subject has KDIGO stage 2 AKI. In another embodiment, the subject has KDIGO stage 3 AKI.

In one embodiment, the subject is determined to have an elevated or high risk of persistent KDIGO stage 2 or 3 AKI.

In another embodiment, the subject is determined to have an elevated or high risk of persistent KDIGO stage 3 AKI.

In one embodiment, the method comprises determining that the subject has an elevated or high risk of developing a persistent KDIGO stage 3 AKI within 48 hours of the time at which the urine sample is obtained, wherein the duration of the persistent KDIGO stage 3 AKI comprises a 72-hour period with a minimum KDIGO stage of KDIGO 3.

In one embodiment, the subject is in an intensive care unit.

In one embodiment, the method further comprises contacting the urine sample with a binding reagent that binds to the CCL14. In one embodiment, the binding reagent is an antibody. In one embodiment, the assay is an immunoassay.

In one aspect, a method for assessing an elevated risk for developing persistent acute kidney injury (AKI) in a subject is provided, the method comprising: (a) performing an assay to detect a level of C—C motif chemokine ligand 14 (CCL14) in a urine sample obtained from the subject; and (b) correlating the assay result to an elevated risk for the subject developing persistent AKI by comparing the assay result to a CCL14 threshold concentration of about 1.3 ng/ml; wherein the correlation is used as a rule-in test for the elevated risk for the subject developing persistent AKI when the assay result is above the CCL14 threshold concentration of about 1.3 ng/ml, or wherein the correlation is used as a rule-out test for the subject having the elevated risk of developing persistent AKI when the assay result is below the CCL14 threshold concentration of about 1.3 ng/ml.

In another aspect, a method for assessing a high risk for developing persistent acute kidney injury (AKI) in a subject is provided, the method comprising: (a) performing an assay to detect a level of C—C motif chemokine ligand 14 (CCL14) in a urine sample obtained from the subject; and (b) correlating the assay result to a high risk for the subject developing persistent AKI by comparing the assay result to a CCL14 threshold concentration of about 13.0 ng/ml; wherein the correlation is used as a rule-in test for the subject having the high risk of developing persistent AKI when the assay result is above the CCL14 threshold concentration of about 13.0 ng/ml, or wherein the correlation is used as a rule-out test for the subject having the high risk of developing persistent AKI when the assay result is below the CCL14 threshold concentration of about 13.0 ng/ml.

In one embodiment, the level of CCL14 detected in the urine sample is above the CCL14 threshold concentration of about 1.3 ng/ml and below a CCL14 threshold concentration of about 13.0 ng/ml.

In one embodiment, the method further comprises seeking further analysis and/or treatment from a nephrologist or specialist.

In some embodiments, the method further comprises treating the subject at elevated or high risk of having persistent AKI with one or more of renal replacement therapy (RRT), withdrawing of compounds that are known to be damaging to the kidney, performing a procedure known to be damaging to the kidney after a delay of at least about 48 hours from obtaining the sample, modifying and/or optimizing diuretic administration, modifying and/or optimizing dosing of renally cleared compounds, and/or administering one or more agents or measured volumes of fluid to restore normal fluid levels, electrolyte levels, or hemodynamics.

In one embodiment, the RRT comprises one or more of continuous RRT, intermittent RRT, hemodialysis, peritoneal dialysis, hemofiltration, and renal transplantation.

In one embodiment, the subject is diagnosed as having AKI.

In one embodiment, the subject has KDIGO stage 1 AKI. In another embodiment, the subject has KDIGO stage 2 AKI. In another embodiment, the subject has KDIGO stage 3 AKI. In one embodiment, the subject is determined to have an elevated or high risk of persistent KDIGO stage 2 or 3 AKI.

In one embodiment, the subject is determined to have an elevated or high risk of persistent KDIGO stage 3 AKI.

In one embodiment, the method comprises determining that the subject has elevated or high risk of developing persistent KDIGO stage 3 AKI within 48 hours of the time at which the urine sample is obtained, wherein the duration of the persistent KDIGO stage 3 AKI comprises a 72-hour period with a minimum KDIGO stage of KDIGO 3.

In one embodiment, the subject is in an intensive care unit.

In one embodiment, the method further comprises contacting the urine sample with a binding reagent that binds to the CCL14. In one embodiment, the binding reagent is an antibody. In one embodiment, the assay is an immunoassay.

In another aspect, a method for assessing a low risk for developing persistent acute kidney injury (AKI) in a subject is provided, the method comprising: (a) performing an assay to detect a level of C—C motif chemokine ligand 14 (CCL14) in a urine sample obtained from the subject; and (b) determining the subject is at low risk for developing persistent AKI based upon the level of CCL14 detected in the urine sample being less than a CCL14 threshold concentration of about 1.3 ng/ml.

In one aspect, a method for assessing an increasing risk for developing persistent acute kidney injury (AKI) in a subject is provided. The method comprises: (a) performing an assay to detect a level of C—C motif chemokine 14 (CCL14) in two or more urine samples obtained from the subject, wherein at least a second urine sample is obtained from the subject within about 72 hours after a first urine sample is obtained; and (b) determining the subject has an increasing risk of persistent AKI based upon the levels of CCL14 detected in the urine samples trending upward.

In one embodiment, the method further comprises (a) performing an assay to detect a level of C—C motif chemokine 14 (CCL14) in two or more urine samples obtained from the subject, wherein at least a second urine sample is obtained from the subject within about 72 hours after a first urine sample is obtained; (b) determining the level of CCL14 detected in the urine sample is low, medium, or high based on a first predetermined threshold concentration of CCL14 and a second predetermined threshold concentration of CCL14, wherein, (i) when the level of C—C motif chemokine 14 in the urine sample is below the first predetermined threshold concentration of CCL14, the level of CCL14 in the urine sample is low; (ii) when the level of CCL14 in the urine sample is above the second predetermined threshold concentration of CCL14, the level of CCL14 in the urine sample is high; or (iii) when the level of CCL14 in the urine sample is between the first predetermined threshold concentration of CCL14 and the second predetermined threshold concentration of CCL14, the level of CCL14 in the urine sample is medium; and (c) correlating the level of CCL14 to an increasing risk that the subject will develop persistent KDIGO stage 1, 2, or 3 AKI when (iv) the level of CCL14 in the first urine sample is low and the level of CCL14 in the second urine sample is medium or high; or (v) the level of CCL14 in the first urine sample is medium and the level of CCL14 in the second urine sample is high.

In some embodiments, the subject has AKI meeting the definition of KDIGO stage 1, the subject has AKI meeting the definition of KDIGO stage 2, or the subject has AKI meeting the definition of KDIGO stage 3.

In some embodiments, the subject is determined to have an increasing risk of the persistent KDIGO stage 2 or 3 AKI. In one embodiment, the subject is determined to have an increasing risk of the persistent KDIGO stage 3 AKI.

In one embodiment, the method comprises, determining that the subject has an increasing risk of developing a persistent KDIGO stage 3 AKI within 48 hours of the time the first urine sample is obtained, wherein the duration of the persistent KDIGO stage 3 AKI comprises a 72-hour period with a minimum KDIGO stage of KDIGO 3.

In one embodiment, the first threshold concentration of CCL14 is less than or equal to 1.3 ng/ml of CCL14. In another embodiment, the second threshold concentration of CCL14 is greater than 13 ng/mL.

In some embodiments, the second urine sample is obtained within about 12 hours of the first urine sample, the second urine sample is obtained about 12 hours after the first urine sample, the second urine sample is obtained within about 24 hours of the first urine sample, the second urine sample is obtained about 24 hours after the first urine sample, the second urine sample is obtained within about 36 hours of the first urine sample, the second urine sample is obtained about 36 hours after the first urine sample, the second urine sample is obtained within about 48 hours of the first urine sample, the second urine sample is obtained about 48 hours after the first urine sample, the second urine sample is obtained within about 72 hours of the first urine sample, or the second urine sample is obtained about 72 hours after the first urine sample.

In one embodiment, the method further comprises obtaining a third urine sample. In one embodiment, the second and third urine samples are obtained within about 12 hours of the first urine sample. In another embodiment, the second and third urine samples are obtained within about 24 hours of the first urine sample. In another embodiment, the second urine sample is obtained at about 12 hours after the first urine sample is obtained and further comprising obtaining a third urine sample at about 24 hours after the first urine sample is obtained.

In one embodiment, the first urine sample and the second urine sample have a low level of CCL14 and the third urine sample has a medium level of CCL14.

In one embodiment, the first urine sample and the second urine sample have a low level of CCL14 and the third urine sample has a high level of CCL14.

In one embodiment, the first urine sample and the second urine sample have a medium level of CCL14 and the third urine sample has a high level of CCL14.

In one embodiment, the first urine sample has a low level of CCL14 and the second urine sample and the third urine sample have a medium level of CCL14.

In one embodiment, the first urine sample has a medium level of CCL14 and the second urine sample and the third urine sample have a high level of CCL14.

In one embodiment, the first urine sample has a low level of CCL14, the second urine sample has a medium level of CCL14, and the third sample has a high level of CCL14.

In one embodiment, the first urine sample has a low level of CCL14, and the second urine sample and the third urine sample have a high level of CCL14.

In one embodiment, the subject is in the intensive care unit.

In one embodiment, the subject is diagnosed as having AKI.

In one embodiment, the subject has had AKI for less than 36 hours before the first urine sample is obtained.

In one embodiment, method further comprises contacting the urine sample with a binding reagent which binds to the CCL14.

In one embodiment, the binding reagent is an antibody.

In one embodiment, the assay is an immunoassay.

In one embodiment, the method further comprises treating the subject at increasing risk of having persistent AKI with one or more of renal replacement therapy (RRT), withdrawing of compounds that are known to be damaging to the kidney, performing a procedure known to be damaging to the kidney after a delay of at least about 48 hours from obtaining the first sample, modifying diuretic administration, modifying dosing of renally cleared compounds, and/or administering one or more agents or measured volumes of fluid to restore normal fluid levels, electrolyte levels, or hemodynamics.

In one embodiment, the RRT comprises one or more of continuous RRT, intermittent RRT, hemodialysis, peritoneal dialysis, hemofiltration, and renal transplantation.

In another aspect, a method for assessing a decreasing risk for developing persistent acute kidney injury (AKI) in a subject is provided. The method comprises: (a) performing an assay to detect a level of C—C motif chemokine 14 (CCL14) in two or more urine samples obtained from the subject, wherein at least a second urine sample is obtained from the subject within about 72 hours after a first urine sample is obtained; and (b) determining the subject has a decreasing risk of persistent AKI based upon the levels of CCL14 detected in the urine samples trending downward.

In one embodiment, the method further comprises: (a) performing an assay to detect a level of C—C motif chemokine 14 (CCL14) in two or more urine samples obtained from the subject, wherein at least a second urine sample is obtained from the subject within about 72 hours after a first urine sample is obtained; (b) determining the level of CCL14 detected in the urine sample is low, medium, or high based on a first predetermined threshold concentration of CCL14 and a second predetermined threshold concentration of CCL14, wherein, (i) when the level of C—C motif chemokine 14 in the urine sample is below the first predetermined threshold concentration of CCL14, the level of CCL14 in the urine sample is low; (ii) when the level of CCL14 in the urine sample is above the second predetermined threshold concentration of CCL14, the level of CCL14 in the urine sample is high; or (iii) when the level of CCL14 in the urine sample is between the first predetermined threshold concentration of CCL14 and the second predetermined threshold concentration of CCL14, the level of CCL14 in the urine sample is medium; and (c) correlating the level of CCL14 to decreasing risk that the subject will develop persistent KDIGO stage 1, 2, or 3 AKI when (iv) the level of CCL14 in the first urine sample is medium and the level of CCL14 in the second urine sample is low; (v) the level of CCL14 in the first urine sample is high and the level of CCL14 in the second urine sample is medium; or (vi) the level of CCL14 in the first urine sample is high and the level of CCL14 in the second urine sample is low.

In one embodiment, the subject has AKI meeting the definition of KDIGO stage 1. In one embodiment, the subject has AKI meeting the definition of KDIGO stage 2. In one embodiment, the subject has AKI meeting the definition of KDIGO stage 3.

In one embodiment, the first threshold concentration of CCL14 is less than or equal to 1.3 ng/ml of CCL14. In another embodiment, the second threshold concentration of CCL14 is greater than 13 ng/mL.

In some embodiments, the second urine sample is obtained within about 12 hours of the first urine sample, the second urine sample is obtained about 12 hours after the first urine sample, the second urine sample is obtained within about 24 hours of the first urine sample, the second urine sample is obtained about 24 hours after the first urine sample, the second urine sample is obtained within about 36 hours of the first urine sample, the second urine sample is obtained about 36 hours after the first urine sample, the second urine sample is obtained within about 48 hours of the first urine sample, the second urine sample is obtained about 48 hours after the first urine sample, the second urine sample is obtained within about 72 hours of the first urine sample, or the second urine sample is obtained about 72 hours after the first urine sample.

In one embodiment, the method further comprises obtaining a third urine sample. In one embodiment, the second and third urine samples are obtained within about 12 hours of the first urine sample. In one embodiment, the second and third urine samples are obtained within about 24 hours of the first urine sample. In one embodiment, the second urine sample is obtained at about 12 hours after the first urine sample is obtained and further comprising obtaining a third urine sample at about 24 hours after the first urine sample is obtained.

In one embodiment, the first urine sample and the second urine sample have a high level of CCL14 and the third urine sample has a medium level of CCL14. In one embodiment, the first urine sample and the second urine sample have a high level of CCL14 and the third urine sample has a low level of CCL14. In one embodiment, the first urine sample and the second urine sample have a medium level of CCL14 and the third urine sample has a low level of CCL14. In one embodiment, the first urine sample has a medium level of CCL14 and the second urine sample and the third urine sample have a low level of CCL14. In one embodiment, the first urine sample has a high level of CCL14 and the second urine sample and the third urine sample have a low level of CCL14. In one embodiment, the first urine sample has a high level of CCL14 and the second urine sample and the third urine sample have a medium level of CCL14. In one embodiment, the first urine sample has a high level of CCL14, the second urine sample has a medium level of CCL14, and the third urine sample has a low level of CCL14.

In one embodiment, the subject is in the intensive care unit.

In one embodiment, the subject is diagnosed as having AKI.

In one embodiment, the subject has had AKI for less than 36 hours before the first urine sample is obtained.

In one embodiment, the method further comprises contacting the urine sample with a binding reagent which binds to the CCL14.

In one embodiment, the binding reagent is an antibody.

In one embodiment, the assay is an immunoassay.

In one embodiment, the method further comprises treating the subject having decreasing risk of persistent AKI by one or more of administering compounds that are known to be damaging to the kidney, performing a procedure known to be damaging to the kidney, modifying and/or optimizing diuretic administration, modifying and or optimizing dosing of renally cleared compounds, and administering one or more agents or measured volumes of fluid to restore normal fluid levels, electrolyte levels, or hemodynamics.

In one embodiment, the method further comprises assessing a risk for developing persistent acute kidney injury (AKI) in a subject by performing an assay to detect a level of C—C motif chemokine 14 (CCL14) in two or more urine samples obtained from the subject, wherein at least a second urine sample is obtained from the subject within about 72 hours after a first urine sample is obtained; and determining the subject has increasing risk of persistent AKI when the levels of CCL14 detected in the urine samples are trending upward, and determining the subject has decreasing risk of persistent AKI when the levels of CCL14 detected in the urine samples are trending downward.

### BRIEF DESCRIPTION OF THE DRAWINGS

FIG. 1 depicts a flow diagram of the pooled analysis of patients from the Ruby and Sapphire studies.

FIG. 2 Illustrates a comparison of CCL14 concentrations in 4 populations.

FIG. 3 depicts risk of the primary endpoint of persistent severe AKI stratified by CCL14 level below, between and above 1.3 and 13 ng/mL.

FIG. 4 illustrates risk of persistent severe AKI stratified by CCL14 level below and above 1.3 ng/mL.

FIGS. 5A-5C illustrate the cumulative incidence of RRT, death and RRT or death within 90 days of enrollment in the Ruby study stratified by CCL14 concentrations below, between and above 1.3 and 13 ng/mL.

FIGS. 6A-6C show cumulative incidence of RRT, death and RRT or death within 90 days of enrollment in the Ruby study stratified by CCL14 concentrations below and above 1.3 ng/mL.

FIG. 7 depicts comparison of CCL14 concentrations in 4 populations.

FIG. 8 illustrates CCL14 trajectories stratified by initial CCL14 category after the diagnosis of moderate to severe AKI in the primary analysis cohort.

FIG. 9 illustrates CCL14 category changes stratified by initial CCL14 category after the diagnosis of moderate to severe AKI.-Sensitivity analysis including patients with <3 CCL14 values.

FIG. 10 illustrates extended measurement of CCL14 over 6-days with first change from initial category.

FIG. 11 illustrates the ability of absolute level of Urinary CCL14 to predict a rolling study endpoint (Persistent severe AKI, RRT or death commencing in subsequent 48 h).

### DETAILED DESCRIPTION

As used herein, “acute kidney injury” or “AKI” is an abrupt (e.g., within about 14 days, such as within about 7 days, within about 72 hours, or within about 48 hours) reduction in kidney function identified by an absolute increase in serum creatinine of greater than or equal to 0.3 mg/dl (≥26.4 μmol/l), a percentage increase in serum creatinine of greater than or equal to 50% (1.5-fold from baseline), or a reduction in urine output (documented oliguria of less than 0.5 ml/kg per hour for at least 6 hours).

As used herein, “persistent AKI” refers to episodes of AKI that persist for at least 48-72 hours before sustained reversal. Reversal of AKI must generally last for a minimum of 48 hours to consider any subsequent episodes of AKI a distinct episode rather than persistence of the original episode. Definitions for various stages of renal injury, including persistent AKI, as well as methods for assessment and treatment may be found in Nat Rev Nephrol. 2017 April; 13 (4): 241-257, which is herein incorporated by reference in its entirety. Persistence of specific stages of AKI (e.g., KDIGO stage 3 AKI) may be defined in a similar manner, wherein a minimum stage of AKI must be maintained for 48-72 hours before sustained recovery from that stage. “Persistent severe AKI” indicates those subjects whose minimum AKI stage during a period of 72 hours is KDIGO stage 3.

“Elevated risk” of persistent AKI means that the level of CCL14 in a sample is greater than a CCL14 threshold concentration of 1.3 ng/ml.

“High risk” of persistent AKI means that the level of CCL14 in a sample is greater than a CCL14 threshold concentration of 13.0 ng/ml.

“Low risk” of persistent AKI means that the level of CCL14 in a sample is less than a CCL14 threshold concentration of 1.3 ng/ml.

In certain embodiments, the level of CCL14 is used as a “rule in” for elevated risk of developing persistent AKI. In these embodiments, the measured level of CCL14 is above a CCL14 threshold concentration of about 1.3 ng/ml.

In certain embodiments, the level of CCL14 is used as a “rule in” for high risk of developing persistent AKI. In these embodiments, the measured level of CCL14 is above a CCL14 threshold concentration of about 13.0 ng/ml.

In certain embodiments, the level of CCL14 is used as a “rule out” for elevated risk of developing persistent AKI. In these embodiments, the measured level of CCL14 is below a CCL14 threshold concentration of about 1.3 ng/ml.

In certain embodiments, the level of CCL14 is used as a “rule out” for high risk of developing persistent AKI. In these embodiments, the measured level of CCL14 is below a CCL14 threshold concentration of about 13.0 ng/ml.

“Increasing risk” of persistent AKI means that the level of CCL14 is trending upward in one or more serial samples obtained from a subject as compared to a reference level (e.g., a baseline level or a threshold level) of CCL14 (e.g., a first CCL14 measurement) obtained from the subject.

“Decreasing risk” of persistent AKI means that the level of CCL14 is trending downward in one or more serial samples obtained from a subject as compared to a reference level (e.g., a baseline level or a threshold level) of CCL14 (e.g., a first CCL14 measurement) obtained from the subject.

“Trending upward” means that a second measurement or level of CCL14 is higher than a first measurement or level of CCL14, e.g. at least about 10% higher, or 20% higher, or 30% higher, or 40% higher, or 50% higher, or 60% higher, or 70% higher, or 80% higher, or 90% higher, or more; or 2-fold higher, or 5-fold higher, or 10-fold higher, or 20-fold higher, or 100-fold higher, or more.

“Trending downward” means that a first measurement or level of CCL14 is higher than a second measurement or level of CCL14, e.g. at least about 10% higher, or 20% higher, or 30% higher, or 40% higher, or 50% higher, or 60% higher, or 70% higher, or 80% higher, or 90% higher, or more; or 2-fold higher, or 5-fold higher, or 10-fold higher, or 20-fold higher, or 100-fold higher, or more.

CCL14 levels can be measured in one or more serial samples obtained from the same subject to monitor for temporal changes in the CCL14 level. For example, CCL14 levels can be measured in 1, 2, 3, 4, 5, 6, 7, 8, 9, 10, or more samples obtained from the same subject. In one embodiment, CCL14 levels are measured in two samples obtained from the same subject. In another embodiment, CCL14 levels are measured in three samples obtained from the same subject. The CCL14 level in the first sample can be used as a baseline level used to monitor changes in CCL14 levels in further samples obtained from the same subject.

After the first sample is obtained, one or more serial samples can be obtained from the subject at any time. The samples can be obtained at about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 72 hours, 7 days, 30 days or more after the first sample is obtained. The samples can be obtained between about 1-3 hours, about 2-4 hours, about 3-5 hours, about 4-6 hours, about 5-7 hours, about 6-8 hours, about 7-9 hours, about 8-10 hours, about 9-11 hours, about 10-12 hours, about 11-13 hours, about 12-14 hours, about 13-15 hours, about 14-16 hours, about 15-17 hours, about 16-18 hours, about 17-19 hours, about 18-20 hours, about 19-21 hours, about 20-22 hours, about 21-23 hours, about 22-24 hours or more after the first sample is obtained. In some embodiments, the samples are obtained within about 1-6 hours, within about 6-12 hours, within about 12-18 hours, within about 18-24 hours, within about 24-36 hours, within about 36-48 hours, within about 48-72 hours, or more after the first sample is obtained.

AKI may be caused by radiocontrast agents (also called contrast media) and other nephrotoxins such as cyclosporine, antibiotics including aminoglycosides and anticancer drugs such as cisplatin typically manifests over a period of days to about a week. Contrast induced nephropathy (CIN, which is AKI caused by radiocontrast agents) is thought to be caused by intrarenal vasoconstriction (leading to ischemic injury) and from the generation of reactive oxygen species that are directly toxic to renal tubular epithelial cells. CIN classically presents as an acute (onset within 24-48 h) but reversible (peak 3-5 days, resolution within 1 week) rise in blood urea nitrogen and serum creatinine.

A commonly reported criterion for defining and detecting AKI is an abrupt (typically within about 2-7 days or within a period of hospitalization) elevation of serum creatinine. Although the use of serum creatinine elevation to define and detect AKI is well established, the magnitude of the serum creatinine elevation and the time over which it is measured to define AKI varies considerably among publications. Traditionally, relatively large increases in serum creatinine such as 100%, 200%, an increase of at least 100% to a value over 2 mg/dL and other definitions were used to define AKI. However, the recent trend has been towards using smaller serum creatinine rises to define AKI. The relationship between serum creatinine rise, AKI and the associated health risks are reviewed in Praught and Shlipak, Curr Opin Nephrol Hypertens 14:265-270, 2005 and Chertow et al, J Am Soc Nephrol 16:3365-3370, 2005, which, with the references listed therein, are hereby incorporated by reference in their entirety. As described in these publications, acute worsening renal function (AKI) and increased risk of death and other detrimental outcomes are now known to be associated with very small increases in serum creatinine. These increases may be determined as a relative (percent) value or a nominal value. Relative increases in serum creatinine as small as 20% from the pre-injury value have been reported to indicate acutely worsening renal function (AKI) and increased health risk, but the more commonly reported value to define AKI and increased health risk is a relative increase of at least 25%. Nominal increases as small as 0.3 mg/dL, 0.2 mg/dL or even 0.1 mg/dL have been reported to indicate worsening renal function and increased risk of death. Various time periods for the serum creatinine to rise to these threshold values have been used to define AKI, for example, ranging from 2 days, 3 days, 7 days, or a variable period defined as the time the patient is in the hospital or intensive care unit. These studies indicate there is not a particular threshold serum creatinine rise (or time period for the rise) for worsening renal function or AKI, but rather a continuous increase in risk with increasing magnitude of serum creatinine rise.

One study (Lassnigg et al, J Am Soc Nephrol 15:1597-1605, 2004) investigated both increases and decreases in serum creatinine. Patients with a mild fall in serum creatinine of −0.1 to −0.3 mg/dL following heart surgery had the lowest mortality rate. Patients with a larger fall in serum creatinine (more than or equal to −0.4 mg/dL) or any increase in serum creatinine had a larger mortality rate. These findings caused the authors to conclude that even very subtle changes in renal function (as detected by small creatinine changes within 48 hours of surgery) seriously effect patient's outcomes. In an effort to reach consensus on a unified classification system for using serum creatinine to define AKI in clinical trials and in clinical practice, Bellomo et al., Crit Care. 8 (4): R204-12, 2004, which is hereby incorporated by reference in its entirety for the RIFLE criteria, proposes the following classifications for stratifying AKI patients:
“Risk”: serum creatinine increased 1.5 fold from baseline OR urine production of <0.5 ml/kg body weight/hr for 6 hours;
“Injury”: serum creatinine increased 2.0 fold from baseline OR urine production <0.5 ml/kg/hr for 12 h;
“Failure”: serum creatinine increased 3.0 fold from baseline OR creatinine ≥4.0 mg/dL (355 μmol/l) with an acute rise of ≥0.5 mg/dl (44 μmol/l) OR urine output below 0.3 ml/kg/hr for 24 h OR anuria for at least 12 hours;

And included two clinical outcomes:
“Loss”: persistent need for renal replacement therapy for more than four weeks.
“ESRD”: end stage renal disease-the need for dialysis for more than 3 months.

These criteria are called the RIFLE criteria, which provide a useful clinical tool to classify renal status. As discussed in Kellum, Crit Care Med. 36: S141-45, 2008 and Ricci et al., Kidney Int. 73, 538-546, 2008, each hereby incorporated by reference in its entirety, the RIFLE criteria provide a uniform definition of AKI which has been validated in numerous studies. As will be understood in the art, RIFLE stage 0 can be used to classify a subject who does not meet the criteria for RIFLE stage R or any more severe RIFLE stage of AKI (i.e. a subject who does not have kidney injury or a subject who has a kidney injury but has not progressed to meeting any of the threshold criteria for RIFLE stage R or more severe RIFLE stages of AKI).

More recently, Mehta et al., Crit Care 11: R31 (doi: 10.1186.cc5713), 2007, hereby incorporated by reference in its entirety, proposes the following similar classifications for stratifying AKI patients (AKIN), which have been modified from RIFLE:
“Stage I”: increase in serum creatinine of more than or equal to 0.3 mg/dL (≥26.4 μmol/L) or increase to more than or equal to 150% (1.5-fold) from baseline OR urine output less than 0.5 mL/kg per hour for more than 6 hours;
“Stage II”: increase in serum creatinine to more than 200% (>2-fold) from baseline OR urine output less than 0.5 mL/kg per hour for more than 12 hours;
“Stage III”: increase in serum creatinine to more than 300% (>3-fold) from baseline OR serum creatinine ≥4.0 mg/dL (>354 μmol/L) accompanied by an acute increase of at least 0.5 mg/dL (44 μmol/L) OR urine output less than 0.3 mL/kg per hour for 24 hours or anuria for 12 hours. As will be understood in the art, AKIN stage 0 can be used to classify a subject who does not meet the criteria for AKIN stage I or any more severe AKIN stage of AKI (i.e. a subject who does not have kidney injury or a subject who has a kidney injury but has not progressed to meeting any of the threshold criteria for AKIN stage I or more severe AKIN stages of AKI).

Likewise, Kidney Disease: Improving Global Outcomes (KDIGO) Acute Kidney Injury Work Group. KDIGO Clinical Practice Guideline for Acute Kidney Injury, Kidney Inter., Suppl. 2012; 2:1-138, incorporated herein by reference in its entirety, refers to both RIFLE and AKIN, and offers the following AKI staging guidelines:

| Stage | Serum creatinine | or | Urine output |
|---|---|---|---|
| 1 | 1.5-1.9 times baseline | | <0.5 ml/kg/h for 6-12 hours |
| | or | | |
| | ≥0.3 mg/dl (≥26.5 mmol/l) increase | | |
| 2 | 2.0-2.9 times baseline | | <0.5 ml/kg/h for ≥12 hours |
| 3 | 3.0 times baseline | | <0.3 ml/kg/h for ≥24 hours |
| | or | | or |
| | Increase in serum creatinine to ≥4.0 mg/dl | | Anuria for ≥12 hours |
| | (≥353.6 mmol/l) | | |
| | or | | |
| | Initiation of renal replacement therapy | | |
| | or | | |
| | In patients <18 years, decrease in eGFR | | |
| | to <35 ml/min per 1.73 m² | | |

As will be understood in the art, KDIGO stage 0 can be used to classify a subject who does not meet the criteria for KDIGO stage 1 or any more severe KDIGO stage of AKI (i.e. a subject who does not have kidney injury or a subject who has a kidney injury but has not progressed to meeting any of the threshold criteria for KDIGO stage 1 or more severe KDIGO stages of AKI).

The CIN Consensus Working Panel (McCollough et al, Rev Cardiovasc Med. 2006;7 (4): 177-197, hereby incorporated by reference in its entirety) uses a serum creatinine rise of 25% to define Contrast induced nephropathy (which is a type of AKI). Although various groups propose slightly different criteria for using serum creatinine to detect AKI, the consensus is that small changes in serum creatinine, such as 0.3 mg/dL or 25%, are sufficient to detect AKI (worsening renal function) and that the magnitude of the serum creatinine change is an indicator of the severity of the AKI and mortality risk.

These classification systems of AKI generally comprise serum creatinine criteria and urine output criteria for each stage. Wherever specified herein, any stage of AKI may be considered equivalent to (i.e, substituted with) any of the individual criteria that qualifies a subject as being at that particular stage of AKI. In some embodiments, the methods disclosed herein may also be used to correlate to a renal status defined by a particular AKI stage (e.g., the likelihood of reaching a particular AKI stage or the likelihood of persistent AKI at a particular stage), wherein the particular AKI stage can be defined by meeting both a serum creatinine criterion that qualifies the subject for that particular stage and a urine output criterion that qualifies a subject for that particular stage. In some embodiments, the particular AKI stage can be defined by meeting all the criteria (i.e. both of all the serum creatinine criteria and all the urine output criteria). All the methods disclosed herein may define stages of AKI according to any of these embodiments, unless stated otherwise. It will be understood in the art, that similarly defined stages of AKI may generally be interchanged with one another as relates to use of the biomarkers disclosed herein, unless dictated otherwise by context. That is, RIFLE stage R, AKIN stage I, and KDIGO stage 1 may generally be interchangeable; RIFLE stage I, AKIN stage II, and KDIGO stage 2 may generally be interchangeable; and RIFLE stage F, AKIN stage III, and KDIGO stage 3 may generally be interchangeable.

Although serial measurement of serum creatinine over a period of days is an accepted method of detecting and diagnosing AKI and is considered one of the most important tools to evaluate AKI patients, serum creatinine is generally regarded to have several limitations in the diagnosis, assessment and monitoring of AKI patients. The time period for serum creatinine to rise to values (e.g., a 0.3 mg/dL or 25% rise) considered diagnostic for AKI can be 48 hours or longer depending on the definition used. Since cellular injury in AKI can occur over a period of hours, serum creatinine elevations detected at 48 hours or longer can be a late indicator of injury, and relying on serum creatinine can thus delay diagnosis of AKI. Furthermore, serum creatinine is not a good indicator of the exact kidney status and treatment needs during the most acute phases of AKI when kidney function is changing rapidly. Some patients with AKI will recover fully, some will need dialysis (either short term or long term) and some will have other detrimental outcomes including death, major adverse cardiac events and chronic kidney disease. Because serum creatinine is a marker of filtration rate, it does not differentiate between the causes of AKI (pre-renal, intrinsic renal, post-renal obstruction, atheroembolic, etc.) or the category or location of injury in intrinsic renal disease (for example, tubular, glomerular or interstitial in origin). Urine output is similarly limited. Knowing these things can be of vital importance in managing and treating patients with AKI.

As used herein, the term “C—C motif chemokine 14” refers to one or more polypeptides present in a biological sample that are derived from the C—C motif chemokine 14 precursor (human sequence: Swiss-Prot Q16627 (SEQ ID NO: 1)):

| | |
|---|---|
| MKISVAAIPF FLLITIALGT KTESSSRGPY HPSECCFTYT | 50 |
| TYKIPRQRIM | |
| DYYETNSQCS KPGIVFITKR GHSVCTNPSD KWVQDYIKDM | 93 |
| KEN | |

The following domains have been identified in C—C motif chemokine 14:

| Residues | Length | Domain ID |
|---|---|---|
| 1-19 | 19 | Signal peptide |
| 20-93 | 74 | C-C motif chemokine 14 |
| 22-93 | 72 | HCC-1(3-74) |
| 23-93 | 71 | HCC-1(4-74 |
| 28-93 | 66 | HCC-1(9-74) |

| | |
|---|---|
| 27 | R→QTGGKPKVVKIQLKLVG in isoform 2 |
| | (SEQ ID NO: 2) |

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

Preferably, an analyte (e.g., CCL14) is measured in a sample. Such a sample may be obtained from a subject or may be obtained from biological materials intended to be provided to the subject. For example, a sample may be obtained from a kidney being evaluated for possible transplantation into a subject, and an analyte measurement used to evaluate the kidney for preexisting damage. Preferred samples are body fluid samples.

The term “body fluid sample” as used herein refers to a sample of bodily fluid obtained for the purpose of diagnosis, prognosis, classification or evaluation of a subject of interest, such as a patient or transplant donor. In certain aspects, such a sample may be obtained for the purpose of determining the outcome of an ongoing condition or the effect of a treatment regimen on a condition, for example, RRT. Preferred body fluid samples include blood (including whole blood, serum, and plasma), cerebrospinal fluid, urine, saliva, sputum, pleural effusions, hemofiltrate, and ultrafiltrate. In addition, one of skill in the art would realize that certain body fluid samples would be more readily analyzed following a fractionation or purification procedure, for example, separation of whole blood into serum or plasma components.

The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “correlating” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy which is highly efficacious and has a low level of risk, few tests are needed because clinicians can accept substantial diagnostic uncertainty. On the other hand, in situations where treatment options are less effective and riskier, clinicians often need a higher degree of diagnostic certainty. Thus, cost/benefit analysis is involved in selecting a diagnostic threshold.

Suitable thresholds may be determined in a variety of ways. For example, one recommended diagnostic threshold for the diagnosis of acute myocardial infarction using cardiac troponin is the 97.5^{th} percentile of the concentration seen in a normal population. Another method may be to look at serial samples from the same patient, where a prior “baseline” result is used to monitor for temporal changes in a biomarker level.

Population studies may also be used to select a decision threshold. Receiver Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when the person tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1-specificity, the ROC graph is sometimes called the sensitivity vs (1-specificity) plot. A perfect test will have an area under the ROC curve of 1.0; a random test will have an area of 0.5. A threshold is selected to provide an acceptable level of specificity and sensitivity.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29:1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.75, even more preferably at least 0.8, still even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1

The present compositions and methods can be used by a clinician to determine treatment options for a subject determined to have an increased risk of persistent AKI or for a subject determined to have a reduced risk of persistent AKI. The treatment options may vary from subject to subject and treatments can be prioritized or deprioritized based on the clinical assessment of the individual subject and other underlying conditions. Non-limiting examples of treatment options for subjects with increased risk for persistent AKI or reduced risk for persistent AKI are disclosed in Chawla L. S., et al., Nat Rev Nephrol. 2017;13 (4): 241-57; Kellum J. A., Critical Care Med., 2015;43 (8): 1785-86; Kashani, K. B., et al., Intensive Care Med. 2020; 46:1036-38, and Kashani K, et al., Critical Care, 2013; 17 (R25): 1-12; each of which is hereby incorporated herein by reference in their entirety. KDIGO Clinical Practice Guideline for Acute Kidney Injury. Kidney Inter., Suppl. 2012; 2:8-12, which is herein incorporated by reference in its entirety, discloses treatments for various stages of AKI. Treatments which are proposed therein for high risk of AKI and AKI stages 1, 2, and 3 include:
1. Discontinue all nephrotoxic agents when possible;
2. Ensure volume status and perfusion pressure;
3. Consider functional hemodynamic monitoring;
4. Monitor serum creatinine and urine output;
5. Avoid hyperglycemia; and
6. Consider alternatives to radiocontrast procedures.

Treatments which are proposed therein for AKI stages 1, 2, and 3 include:
1. Non-invasive diagnostic workup; and
2. Consider invasive diagnostic workup

Treatments which are proposed therein for AKI stages 2, and 3 include:
1. Check for changes in drug dosing;
2. Consider renal replacement therapy (RRT); and
3. Consider ICU admission.

For AKI stage 3, it is also advised therein to avoid subclavian catheters, if possible.

Renal replacement therapy (RRT) is an option for management of patients suffering from renal dysfunction, including AKI, persistent AKI, AKD, or CKD. RRT, as used herein, is interchangeable with kidney replacement therapy (KRT) and includes renal transplant as well as various types of dialysis. Dialysis filters and removes waste products, electrolytes, and water from the body similar to the function of the kidney. Multiple dialysis protocols are in use. The different types of dialysis generally fall within the categories of hemodialysis and peritoneal dialysis. Hemodialysis clears solutes from the blood by diffusion across an artificial membrane using a concentration gradient. Peritoneal dialysis, which uses the peritoneum as a semi-permeable membrane to remove solvents, is also in clinical use. Unlike hemodialysis which directly filters the blood, peritoneal dialysis includes injecting fluid into the peritoneal cavity. The peritoneum acts as a filter and fluid is then removed with accompanying waste products, electrolytes, and excess water. Timing of dialysis has been shown to be relevant to the patient outcome. Reviewed by Pannu N. and Noel Gibney R. T. Ther Clin Risk Manag. 2005;1 (2): 141-50, which is hereby incorporated by reference in its entirety. More specific dialysis procedures include intermittent renal replacement therapies (IRRTs) and continuous renal replacement therapies (CRRTs). IRRTs include intermittent hemodialysis, intermittent hemofiltration, and intermittent hemodiafiltration. CRRTs include continuous hemofiltration and continuous hemodiafiltration. There are also hybrid dialysis protocols called prolonged intermittent renal replacement therapies (PIRRTs). These include sustained low-efficiency dialysis (SLED) and extended-duration dialysis (EDD). Some types may be performed at the subject's home or during travel while some require a clinical setting with the assistance of healthcare professionals.

In some embodiments, the treatment options include accelerating initiation of RRT or other treatments or procedures, referring the patient for further analysis to determine the appropriate treatment regimen, withdrawing of compounds that are known to be damaging to the kidney, performing a procedure known to be damaging to the kidney after a delay of at least about 48 hours from obtaining the sample, modifying and/or optimizing diuretic administration, modifying and/or optimizing dosing of renally cleared compounds, and/or administering one or more agents or measured volumes of fluid to restore normal fluid levels, electrolyte levels, or hemodynamics.

In some cases, the subject may seek further analysis and/or treatment from a nephrologist or specialist. In one embodiment the further analysis includes performing additional tests. Non-limiting examples of analyses that may be performed include evaluation of urine sediment, proteinuria, and renal ultrasound; kidney biopsy; therapeutic drug monitoring or optimizing; hemodynamic monitoring and/or optimizing; performing an assessment of AKI etiology for persistent AKI; and further analysis to diagnose and/or treat rarer causes of AKI (interstitial nephritis, tumor lysis syndrome, thrombotic thrombocytopeniaurpura, and cholesterol embolization syndrome).

### EXAMPLES

### Example 1

### Introduction

In the Ruby study, we enrolled critically ill patients within 36 hours of clinical diagnosis of Kidney Disease Improving Global Outcomes (KDIGO) Stage 2 (moderate) or 3 (severe) AKI. We reported on the performance of urinary C—C motif chemokine ligand 14 (CCL14) for the prediction of persistent (>72 hours) Kidney Disease Improving Global Outcomes (KDIGO) Stage 3 (severe) AKI. In this pre-planned secondary analysis of the Ruby study, we report on the performance of a standardized clinical assay, the NEPHROCLEAR™ CCL14 test, along with the derivation and operating characteristics of two distinct cutoff values on the CCL14 receiver operating characteristic (ROC) curve for prediction of persistent severe AKI. We chose one cutoff for high sensitivity and another for high specificity. We compared rates of renal replacement therapy (RRT) or death across CCL14 strata defined by the cutoffs over the first 90 days post-enrollment. We also evaluated CCL14 cutoffs together with clinical variables from the Ruby study and assessed whether there was added value of the biomarker in a clinical model. Finally, we examined the distribution of urinary CCL14 levels relative to the cutoffs in reference populations comprising apparently healthy subjects and subjects with stable chronic comorbidities.

### Methods

### Subjects

The Ruby study and the reference population study have both been described previously (Hoste E, et al. Intensive Care Med; 2020; 46:943-953 and Chindarkar N S, et al. Clinica Chimica Acta; 2016; 452:32-37, both of which are hereby incorporated by reference in their entirety. Ruby enrolled adult ICU patients with established KDIGO stage 2-3 AKI across 21 sites in Europe and the United States from June 2013 to May 2014. Patients were excluded if they had a prior kidney transplant, were receiving or were in imminent need of RRT, were receiving comfort measures only, or had known infection with human immunodeficiency virus or active hepatitis. The reference population study enrolled two cohorts of adult subjects at six sites from April 2012 to November 2012: (A) apparently healthy subjects and (B) subjects with pre-specified stable chronic conditions without acute illness. Protocols for both studies were approved by investigational review boards or ethics committees as required by each participating site with all subjects (or their proxies) providing written informed consent.

### Study Endpoint

The primary endpoint of the Ruby study was persistent severe AKI, defined as KDIGO stage 3 AKI for at least 72 consecutive hours. Patients who received RRT or died prior to achieving 72 hours in stage 3 AKI were considered endpoint positive. Patients who were in stage 2 AKI at the time of enrollment who received RRT or progressed to persistent severe AKI starting within 48 hours were also considered endpoint positive. Reference serum creatinine was determined by expert adjudication blinded to the biomarker results, as previously described. Secondary endpoints included RRT initiation, death, and the composite of RRT initiation or death within 90 days.

### Sample Collection and Testing

In both the Ruby and reference population cohorts, urine samples were collected at enrollment and centrifuged. Supernatants were flash frozen within 2 hours of collection and stored at ≤−70° C. Thawed samples were analyzed for CCL14 using the NEPHROCLEAR™ CCL14 Test on the Astute140® Meter (Astute Medical, San Diego, CA) by operators blinded to the clinical data. Concentration results for the NEPHROCLEAR™ CCL14 Test are traceable to reference material that contains defined mass of the CCL14 protein in accordance with EN ISO 17511.

### Cutoff Selection

Two cutoffs on the urinary CCL14 ROC curve were selected for risk assessment for development of persistent severe AKI. A lower CCL14 concentration cutoff was chosen to achieve high sensitivity with reasonable specificity to enable early recognition of the majority of patients who subsequently developed persistent severe AKI. This cutoff was chosen to identify patients who would be candidates for actions recommended as increasing in priority by the KDIGO Clinical Practice Guideline as risk increases for adverse consequences of AKI. The types of actions considered in determining the appropriate balance between sensitivity and specificity included consultation from experts (nephrology, pharmacy, intensive care etc.), additional diagnostic work up and prioritized evaluation of need for higher intensity monitoring (including hemodynamic/fluid monitoring and possible ICU admission) or kidney replacement therapy. In selecting the cutoff, we assumed the test will not be used in a stand-alone manner as the sole basis for initiating invasive procedures, which should be ordered only after careful consideration of all clinical and lab test information, to ensure the potential benefits outweigh the risks. A second, higher concentration, cutoff was selected to enable identification of patients who will develop persistent severe AKI with high specificity. This high specificity cutoff was selected to identify the subgroup of patients who are at the highest risk of persistent severe AKI and thus have the most urgent need for evaluation to receive actions recommended by the KDIGO Clinical Practice Guideline. Importantly, values between these cutoffs are not “indeterminant” nor do values in this range represent a “grey zone”. Instead, this portion of the ROC curve reflects a transition from high sensitivity to high specificity.

### Statistical Analysis

Operating characteristics including sensitivity, specificity, negative and positive predictive values, and negative and positive likelihood ratios for the primary endpoint were calculated throughout the range of CCL14 concentrations to assess the clinical performance at different cutoffs. Differences in CCL14 concentrations among healthy subjects, those with chronic conditions, and Ruby subjects who did and did not develop the primary endpoint were presented in box and whisker plots. The Kruskal-Wallis test was used to detect differences in the concentrations among the four groups. Risk and relative risk for developing persistent severe AKI were calculated by dividing the cohort with the cutoffs. The reference stratum for relative risk consisted of those patients with CCL14 concentrations less than or equal to the lower (high sensitivity) cutoff. The Cochran-Armitage test was used to determine trend across risk strata defined by the cutoffs. The cumulative incidence curves for RRT initiation, death, and composite of RRT initiation or death within 90 days were estimated using the Kaplan-Meier method, and log-rank test was used to compare groups defined by two cutoffs. To examine whether urinary CCL14 stratified by the two cutoffs improved risk prediction beyond clinical variables alone, a reference logistic regression model was constructed as described previously. When analyzing CCL14 as a categorical variable in regression analyses, CCL14≤1.3 ng/ml (the lower cutoff) was used as the reference level. Integrated discrimination improvement (IDI) and category-free net reclassification (cfNRI) were used to assess the enhancement of the risk prediction by CCL14. Continuous, dichotomous, and polytomous baseline variables were compared across CCL14 strata by the Kruskal-Wallis, Cochran-Armitage, and Fisher's exact tests, respectively. Confidence intervals for sensitivity, specificity, positive and negative predictive value were calculated by the Clopper-Pearson exact method, while those for positive and negative likelihood ratio, risk, and relative risk were computed by the asymptotic method (normal approximation). Two-sided p values less than 0.05 were considered statistically significant. Statistical analyses were performed using R 4.0.2 (R Foundation for Statistical Computing. Vienna, Austria), and IDI and cfNRI were calculated using the “Hmisc” package.

### Results

A cutoff value for urinary CCL14 of 1.3 ng/ml was determined to achieve high sensitivity ((91% (95% CI: 84%-96%)) and a cutoff of 13 ng/ml was found to achieve high specificity (93% (89%-96%)) with the range in between reflecting a transition from high sensitivity to high specificity. Baseline characteristics for all Ruby study patients at enrollment are shown in Table 1 stratified by the two cutoffs (≤1.3, 1.3 to 13 and >13 ng/mL). Patients with higher CCL14 levels (>13 ng/ml) were less likely to have a history of coronary artery disease, but more likely to be admitted to the ICU for respiratory failure or sepsis. Two hundred and eleven (62.9%) patients had a CCL14 >1.3 ng/ml, while 54 (16.1%) had a value >13 ng/ml. While there was no difference in the retrospectively adjudicated pre-admission baseline serum creatinine across the CCL14 strata, patients with a CCL14 concentration >13 ng/ml had significantly higher serum creatinine at study enrollment compared to those with a CCL14≤1.3 ng/mL (p<0.001 between CCL14≤1.3 and CCL14 >13). Using the previously described, retrospective adjudication of baseline serum creatinine, 55 patients (16.4% of the entire cohort) did not meet criteria for stage 2-3 AKI at the time of enrollment. Patients with higher CCL14 levels had more severe AKI at the time of study enrollment (p<0.001). Table 2 provides the baseline characteristics for the cohort dichotomized by a CCL14 concentration higher or lower than 1.3 ng/ml.

Enrollment serum creatinine (SCr) was higher in those with elevated CCL14 concentrations and provided an AUC of 0.81 for the primary endpoint of persistent severe AKI. Adding urine CCL14 as a categorical variable stratified by the two cutoffs to enrollment SCr led to a significant increase in the AUC to 0.85 (p=0.02). We also compared the performance of 24-hour pre-enrollment urine output and CCL14 to predict the primary outcome. Tables 3A and 3B demonstrate the poor performance of urine output to predict the primary outcome (AUC=0.63) as well as its non-significant odds ratio (95% CI) in a multi-variate model 0.76 (0.57-1.00); P-0.053. The low cutoff of 1.3 ng/mL was selected to achieve high sensitivity (91%), i.e., to identify most of the subjects who progress to persistent severe AKI. Similarly, the high cutoff of 13 ng/ml was selected to achieve high specificity (93%), i.e., to identify most of the subjects who will not progress to persistent severe AKI.

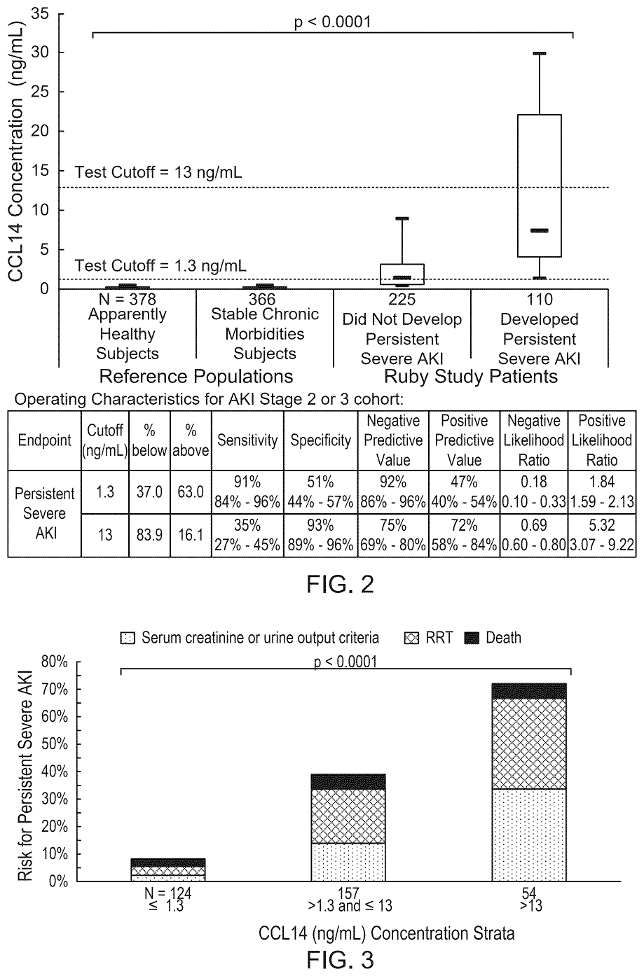

The operating characteristics from the Ruby study for the two cutoffs and position of the cutoffs relative to the distributions of urinary CCL14 levels in the Ruby study and reference population cohorts are shown in FIG. 2. These cohorts are healthy (378), chronic conditions without acute illness (366), Ruby-did not develop persistent severe AKI (225), and Ruby-developed persistent severe AKI (110). The bottom and top whiskers in the drawing represent the 10^{th} and 90^{th} percentiles of the CCL14 concentrations in that group, respectively. The bottom and top boxes represent the 1^{st} and 3^{rd} quartiles, respectively. The middle bar is the median. The horizontal dashed lines correspond to the 1.3 and 13 ng/mL cutoffs. P-value computed using the Kruskal-Wallis test <0.0001. The cutoff at 13 ng/ml is higher than the vast majority (93%) of CCL14 values from Ruby study patients who did not develop persistent severe AKI and has a high positive predictive value of 72% (with a negative predictive value of 75%). Both cutoffs are substantially above the urinary CCL14 ranges for apparently healthy and stable chronic comorbidities reference cohorts. Different cutoffs can be selected to achieve different balance of sensitivity and specificity according to clinical need. For example, Table 4 provides the operating characteristics for urinary CCL14 values from 0.2 to 30 ng/ml in the Ruby study population.

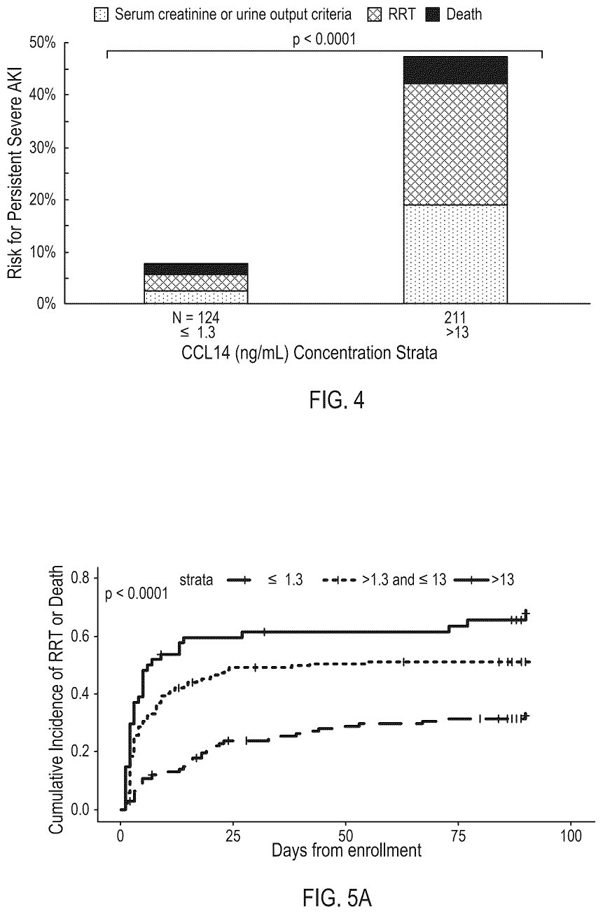

FIG. 3 shows the risk of developing persistent severe AKI across the 3 strata of CCL14 levels defined by the two cutoffs. Within each CCL14 stratum, the individual components of the composite endpoint are displayed. Endpoint components are shown based on the first criterion (e.g. serum creatinine or urine output) that was met. The endpoint was ascertained within 5 days of enrollment. The shading in each stratum shows the relative contribution of patients who met the persistent severe AKI composite endpoint by starting renal replacement therapy (RRT), death or persistently (≥72 h) elevated serum creatinine or oliguria. Risk for the composite endpoint increased significantly with increasing CCL14 level, and the individual components, RRT and persistently elevated serum creatinine or oliguria were stratified similarly (p<0.0001 for both). Increase in risk for death, as a component of the primary endpoint, was not statistically significant (p=0.13). Patients with a CCL14 concentration between 1.3 and 13 ng/mL were 4.8 (2.6-9.0) times as likely to develop persistent severe AKI compared to those with CCL14≤1.3, while those with CCL14 >13 ng/ml were 9.0 (4.8-17) times as likely to develop persistent severe AKI. Risk of developing persistent severe AKI across the 2 strata of CCL14 levels defined by the 1.3 ng/ml cutoff are shown in FIG. 4. In reference to FIG. 4, within each CCL14 stratum the individual components of the composite endpoint are displayed. Endpoint components are shown based on the first criterion (e.g. serum creatinine or urine output) that was met. The endpoint was ascertained within 5 days of enrollment.

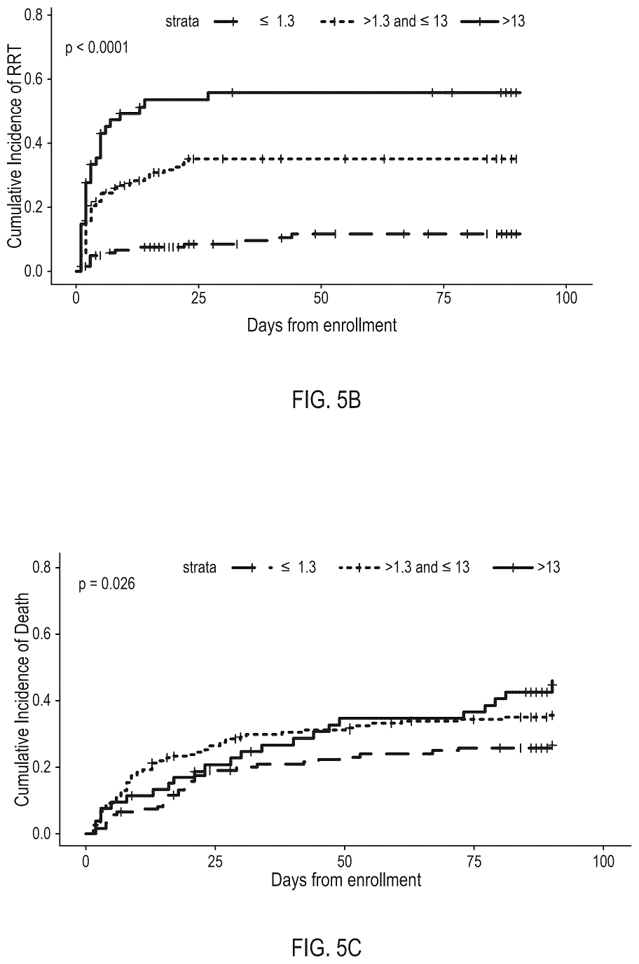

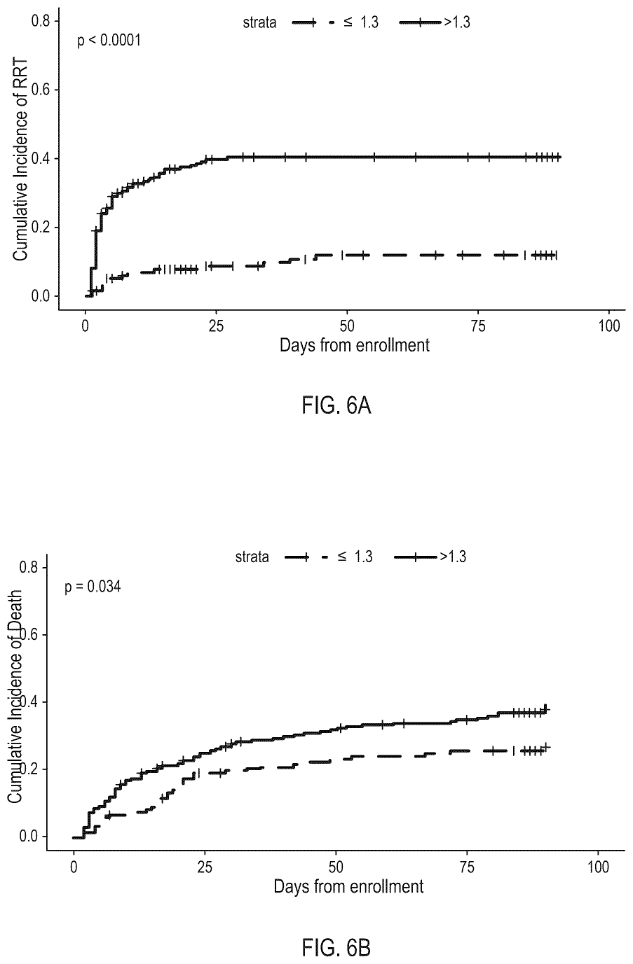

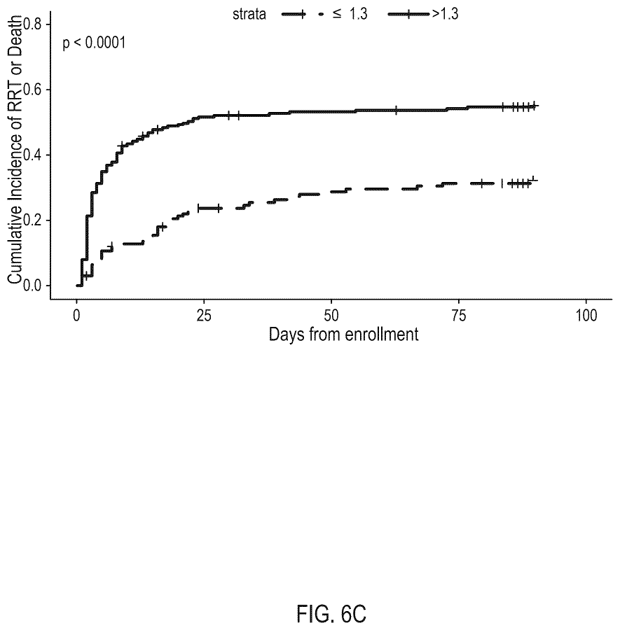

FIGS. 5A-5C illustrate the individual and combined rates of RRT and death in the cohort across CCL14 strata over the first 90 days post-enrollment. In the graphs presented in FIGS. 5A, 5B, and 5C, the number of patients with CCL14 concentrations below, between and above 1.3 and 13 ng/ml are 124, 157, and 54, respectively are shown. The log rank test for trend was used to compute the p-value for differences among the strata. Higher CCL14 values were associated with an increased risk of both death and RRT as well as the composite of the 2 (log rank p<0.001). These findings persisted when the cohort was dichotomized at a CCL14 of 1.3 ng/ml (FIGS. 6A-6C). Referring to the data illustrated in FIGS. 6A, 6B, and 6C, the number of patients with CCL14 concentrations below and above 1.3 ng/ml are 124 and 211, respectively. The log rank test was used to compute the p-value for the differences between the strata. Table 5 demonstrates the median time (days from enrollment) for subjects to receive RRT or die.

In order to evaluate the biomarker cutoffs in comparison with clinical variables we performed a multivariable logistic regression analysis with persistent AKI as the endpoint with and without CCL14 as a covariate (Table 6). The results demonstrate that CCL14 concentrations were significant even after accounting for clinical variables previously shown to be associated with persistent AKI. We found that the use of the 2 cutoffs added value to the clinical model using IDI and cfNRI analysis (Table 7). The addition of these CCL14 concentrations to the model significantly (p=0.02) increased the AUC (95% CI) from 0.86 (0.82-0.90) to 0.88 (0.85-0.92).

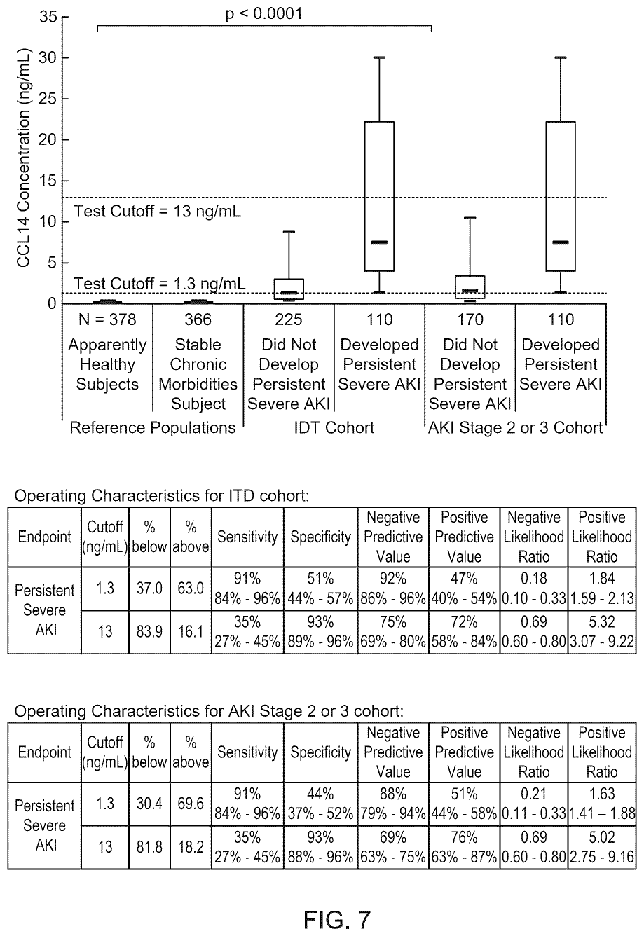

Finally, as described above we retrospectively adjudicated baseline creatinine for all patients. Using this retrospective baseline creatinine to determine AKI stage, 55 patients were determined not to have Stage 2 or 3 AKI at enrollment. 37 of these 55 patients (67%) had Stage 2-3 AKI within the 24 hours prior to enrollment, but their AKI Stage improved. We conducted a sensitivity analysis excluding these 55 patients. Table 8 demonstrates that excluding these patients did not significantly change the odds ratio of either strata of CCL14 concentration (compared to Table 6). Additionally, FIG. 7 displays the operating characteristics for those subjects adjudicated to have Stage 2 or 3 at enrollment for the two cutoffs and position of the cutoffs relative to the distributions of urinary CCL14 levels in this cohort and reference population cohorts. The operating characteristic remain similar to those displayed in the intention-to-diagnose (ITD) cohort (FIG. 2). The cohorts shown in FIG. 7 are healthy (378), chronic conditions without acute illness (366), Ruby Intention-to-Diagnose (ITD) Full cohort (335, 225 of whom did not develop persistent severe AKI), and those adjudicated to have Stage 2 or 3 at enrollment (280, 170 of whom did not develop persistent severe AKI). The bottom and top whiskers represent the 10^{th} and 90^{th} percentiles of the CCL14 concentrations in that group, respectively. The bottom and top boxes represent the 1^{st} and 3^{rd} quartiles, respectively. Middle bar is the median. The horizontal dashed lines correspond to the 1.3 and 13 ng/mL cutoffs. P-value computed using the Kruskal-Wallis test <0.0001.

### DISCUSSION

We have derived and characterized two cutoffs for urinary CCL14 using a standardized clinical assay to aid in clinical risk assessment for the development of persistent severe AKI. Assay standardization is essential for clinical implementation and is required before cutoffs can be specified. These cutoffs were determined by optimizing the operating characteristics (sensitivity, specificity, negative and positive predictive values) based on the intended clinical use of the NEPHROCLEAR™ CCL14 test. A high sensitivity cutoff at 1.3 ng/ml CCL14 can be used to identify the vast majority of patients who will develop persistent severe AKI, and who thus are candidates for actions recommended as increasing in priority by the KDIGO Clinical Practice Guideline as risk increases for adverse consequences of AKI. A high specificity cutoff at 13 ng/ml can be used to identify the highest risk patients who are in most urgent need of evaluation for further intervention. Clinical relevance of these two cutoffs is demonstrated by their ability to stratify risk for adverse events (RRT or death) over 90 days and to add significant predictive information over clinically available information. Importantly, while CCL14 has already been validated as a biomarker for persistent AKI this is the first report validating specific cutoffs using a standardized CCL14 assay developed for routine clinical use. This work represents an important step in framing the clinical utility of CCL14 for the identification of those at risk for persistent severe AKI.

We have demonstrated that urinary CCL14 provides significant information about the likelihood of persistent AKI that cannot be inferred from existing laboratory tests such as serum creatinine or clinical variables alone. In our multivariable model (Table 6) CCL14 remained statistically significant with elevated values being associated with 10.4 times the odds of persistent AKI. Similarly, the IDI and cfNRI were both statistically significant (p<0.001 for both) (Table 7). Therefore, the test provides important new information that can be used in conjunction with existing laboratory tests and clinical assessment to provide the most comprehensive view of a patient's acute kidney status and likely course without further intervention. The high negative predictive value (92%) at the cutoff of 1.3 ng/mL ensures that almost all patients who test negative will not develop persistent AKI. Additionally, a value above 1.3 ng/mL relates to a nearly 1 in 2 risk of developing persistent AKI (positive predictive value (PPV) of 48%). At the higher cutoff (>13 ng/mL), the PPV increased further to 72% which is in line with the PPV performance of other biomarkers such as B-type-natriuretic-peptide (100 μg/mL had PPV 79% for acute heart failure) and high-sensitivity troponin I (>30 pg/mL had a PPV of 75% for acute coronary syndrome).

The ability to detect persistence early in a course of severe AKI has the potential to make a major clinical impact in the care of critically ill patients. There is an increasing body of literature demonstrating that patients with established AKI do not often receive guideline-based care. In multiple large scale retrospective single center cohorts and one multicenter, international cohort, patients with known KDIGO AKI continued to receive nephrotoxins (including NSAIDS and aminoglycosides) and experienced persistent hypotension (e.g. mean arterial pressures below 55 mmHg), even in the setting of established Stage 2 AKI. One of the most significant barriers to delivering these recommended clinical actions is that most AKI resolves spontaneously or with initial clinical management and it is quite difficult to know which patients will require discontinuation of nephrotoxic drugs or a more intensive assessment of hemodynamics/volume status. We submit that a tool like CCL14 that can better predict persistent severe AKI can serve as a tool to determine which patients need additional kidney focused care beyond early management as it stands to reason that those destined for persistent severe AKI are most likely to benefit from strict adherence to kidney care bundles. For example, consider an ICU patient who develops early Stage 1 AKI. A clinician reviews their medication list and assesses their volume status. They determine that the patient needs an additional bolus of balanced intravenous fluids, adjustment of vancomycin dosing based on therapeutic monitoring and to have piperacillin-tazobactam changed to cefepime. Now consider if this patient progresses to Stage 2 AKI, knowing that serum creatinine changes often reflect injuries that have already occurred. The clinician will therefore be uncertain as to whether additional management changes are needed. If CCL14 is measured and is >1.3 ng/ml then it may be appropriate to consider more aggressive clinical maneuvers (more intensive hemodynamic monitoring, stopping/changing other medications or consulting nephrology). However, if the CCL14 is ≤1.3 ng/ml then clinicians may be inclined to continue present management and make no further interventions.

Another clinical utilization of CCL14 could be around the initiation of RRT in the ICU. The Ruby cohort CCL14 results stratify patients with respect to initiation of RRT in the short term (FIG. 3) or longer (FIGS. 5A-5C). While laboratory tests alone are not expected to indicate which patient should or should not receive RRT, CCL14 may identify patients for whom additional clinical evaluation is appropriate in conjunction with medical history, physical examination, fluid balance, blood chemistries, etc.

In summary we have validated a standardized clinical assay for CCL14, the NEPHROCLEAR™ CCL14 test, and derived and characterized cutoffs for CCL14 using this test that allow for the identification of patients at elevated and high risk for the development of severe persistent AKI. These results have immediate utility in helping to guide patient care and may facilitate future clinical trials.

TABLE 1
Baseline characteristics for all patients and stratified by the urinary CCL14 cutoffs at 1.3 and 13 ng/mL.

| | | | | | >1.3 and ≤13 | | | | |
|---|---|---|---|---|---|---|---|---|---|
| | All patients | | ≤1.3 ng/mL | | (ng/mL) | | >13 ng/mL | | p value |
| Patients | 335 | | 124 | | 157 | | 54 | | |
| Male | 209 | (62.4%) | 81 | (65.3%) | 96 | (61.1%) | 32 | (59.3%) | 0.389 |
| Age¹ (years) | 64 | (55-73) | 63 | (55-71) | 65 | (56-73) | 62 | (52-72) | 0.349 |
| BMI¹ (kg/m²) | 29 | (25-35) | 30 | (26-36) | 29 | (25-36) | 28 | (24-32) | 0.121 |
| Race | | | | | | | | | |
| Black or African American | 35 | (10.4%) | 17 | (13.7%) | 12 | (7.6%) | 6 | (11.1%) | 0.066 |
| Other/Unknown | 17 | (5.1%) | 2 | (1.6%) | 10 | (6.4%) | 5 | (9.3%) | |
| White or Caucasian | 283 | (84.5%) | 105 | (84.7%) | 135 | (86.0%) | 43 | (79.6%) | |
| Chronic comorbidities | | | | | | | | | |
| CKD | 59 | (17.6%) | 17 | (13.7%) | 31 | (19.9%) | 11 | (20.4%) | 0.192 |
| Diabetes | 110 | (32.8%) | 42 | (34.1%) | 53 | (34.0%) | 15 | (28.3%) | 0.524 |
| CHF | 73 | (21.8%) | 32 | (26.4%) | 32 | (20.4%) | 9 | (16.7%) | 0.116 |
| CAD | 118 | (35.2%) | 56 | (45.5%) | 49 | (31.6%) | 13 | (24.5%) | 0.003 |
| Hypertension | 228 | (68.1%) | 88 | (71.5%) | 109 | (69.4%) | 31 | (57.4%) | 0.096 |
| COPD | 54 | (16.1%) | 16 | (13.0%) | 33 | (21.2%) | 5 | (9.3%) | 0.968 |
| Cancer | 85 | (25.4%) | 23 | (18.5%) | 46 | (29.5%) | 16 | (29.6%) | 0.052 |
| Reason for ICU admission | | | | | | | | | |
| Respiratory | 96 | (28.7%) | 24 | (19.4%) | 53 | (33.8%) | 19 | (35.2%) | 0.009 |
| Surgery | 105 | (31.3%) | 41 | (33.1%) | 50 | (31.8%) | 14 | (25.9%) | 0.393 |
| Cardiovascular | 149 | (44.5%) | 54 | (43.5%) | 69 | (43.9%) | 26 | (48.1%) | 0.622 |
| Sepsis | 75 | (22.4%) | 19 | (15.3%) | 39 | (24.8%) | 17 | (31.5%) | 0.010 |
| Neurological | 17 | (5.1%) | 8 | (6.5%) | 6 | (3.8%) | 3 | (5.6%) | 0.606 |
| Trauma | 7 | (2.1%) | 3 | (2.4%) | 2 | (1.3%) | 2 | (3.7%) | 0.800 |
| Other | 109 | (32.5%) | 39 | (31.5%) | 53 | (33.8%) | 17 | (31.5%) | 0.897 |
| Vasopressors² | 213 | (63.6%) | 81 | (65.3%) | 102 | (65.0%) | 30 | (55.6%) | 0.291 |
| Diuretics² | 181 | (54.0%) | 76 | (61.3%) | 81 | (51.6%) | 24 | (44.4%) | 0.026 |
| Fluid balance^{1, 3} (mL) | 3271 | (1285-6422) | 2267 | (365-4219) | 4282 | (1932-7419) | 3420 | (1554-7447) | <0.001 |
| Days from ICU admission to enrollment¹ | 1.1 | (0.7-2.2) | 1.4 | (0.8-2.8) | 1.1 | (0.7-1.9) | 1.1 | (0.6-1.9) | 0.110 |
| Mechanical ventilation | 187 | (55.8%) | 65 | (52.4%) | 96 | (61.1%) | 26 | (48.1%) | 0.991 |
| Baseline serum creatinine¹ (mg/dL) | 1.0 | (0.8-1.2) | 1.0 | (0.7-1.2) | 1.0 | (0.8-1.3) | 1.0 | (0.8-1.3) | 0.107 |
| Enrollment serum creatinine¹ (mg/dL) | 2.4 | (1.7-3.3) | 1.7 | (1.3-2.5) | 2.8 | (1.9-3.6) | 3.2 | (2.4-4.2) | <0.001 |
| Enrollment KDIGO Stage | | | | | | | | | |
| No AKI | 18 | (5.4%) | 16 | (12.9%) | 2 | (1.3%) | 0 | (0.0%) | |
| Stage 1 | 37 | (11.0%) | 23 | (18.5%) | 11 | (7.0%) | 3 | (5.6%) | <0.001 |
| Stage 2 | 169 | (50.4%) | 67 | (54.0%) | 86 | (54.8%) | 16 | (29.6%) | |
| Stage 3 | 111 | (33.1%) | 18 | (14.5%) | 58 | (36.9%) | 35 | (64.8%) | |
| Enrollment non-renal APACHE III score¹ | 54 | (43-71) | 48 | (37-64) | 55 | (46-72) | 61 | (43-81) | 0.003 |

¹Median (interquartile range)
²Vasopressors and diuretics are defined as any use from 3 days before through Day 1 (day of study enrollment).
³Fluid balance is cumulative from the day prior to through the day of enrollment.

TABLE 2
Table of baseline characteristics with patients dichotomized
by CCL14 concentrations above and at or below 1.3 ng/mL.

| | All patients | ≤1.3 ng/mL | >1.3 ng/mL | p value |
|---|---|---|---|---|
| Patients | 335 | 124 | 211 | |
| Male | 209 (62.4%) | 81 (65.3%) | 128 (60.7%) | 0.416 |
| Age¹ (years) | 64 (55-73) | 63 (55-71) | 64 (55-73) | 0.473 |
| BMI¹ (kg/m²) | 29 (25-35) | 30 (26-36) | 28 (25-35) | 0.131 |
| Race | | | | |
| Black or African American | 35 (10.4%) | 17 (13.7%) | 18 (8.5%) | |
| Other/Unknown | 17 (5.1%) | 2 (1.6%) | 15 (7.1%) | 0.030 |
| White or Caucasian | 283 (84.5%) | 105 (84.7%) | 178 (84.4%) | |
| Chronic comorbidities | | | | |
| CKD | 59 (17.6%) | 17 (13.7%) | 42 (20.0%) | 0.181 |
| Diabetes | 110 (32.8%) | 42 (34.1%) | 68 (32.5%) | 0.810 |
| CHF | 73 (21.8%) | 32 (26.4%) | 41 (19.4%) | 0.168 |
| CAD | 118 (35.2%) | 56 (45.5%) | 62 (29.8%) | 0.004 |
| Hypertension | 228 (68.1%) | 88 (71.5%) | 140 (66.4%) | 0.333 |
| COPD | 54 (16.1%) | 16 (13.0%) | 38 (18.1%) | 0.281 |
| Cancer | 85 (25.4%) | 23 (18.5%) | 62 (29.5%) | 0.028 |
| Reason for ICU admission | | | | |
| Respiratory | 96 (28.7%) | 24 (19.4%) | 72 (34.1%) | 0.004 |
| Surgery | 105 (31.3%) | 41 (33.1%) | 64 (30.3%) | 0.627 |
| Cardiovascular | 149 (44.5%) | 54 (43.5%) | 95 (45.0%) | 0.821 |
| Sepsis | 75 (22.4%) | 19 (15.3%) | 56 (26.5%) | 0.021 |
| Neurological | 17 (5.1%) | 8 (6.5%) | 9 (4.3%) | 0.442 |
| Trauma | 7 (2.1%) | 3 (2.4%) | 4 (1.9%) | 0.713 |
| Other | 109 (32.5%) | 39 (31.5%) | 70 (33.2%) | 0.809 |
| Vasopressors² | 213 (63.6%) | 81 (65.3%) | 132 (62.6%) | 0.640 |
| Diuretics² | 181 (54.0%) | 76 (61.3%) | 105 (49.8%) | 0.042 |
| Fluid balance over the 72 hours prior | 3271 (1285-6422) | 2267 (365-4219) | 4244 (1851-7430) | <0.001 |
| to enrollment^{1, 3} (mL) | | | | |
| Days from ICU admission to enrollment¹ | 1.1 (0.7-2.2) | 1.4 (0.8-2.8) | 1.1 (0.7-1.9) | 0.037 |
| Mechanical ventilation | 187 (55.8%) | 65 (52.4%) | 122 (57.8%) | 0.363 |
| Baseline serum creatinine¹ (mg/dL) | 1.0 (0.8-1.2) | 1.0 (0.7-1.2) | 1.0 (0.8-1.3) | 0.036 |
| Enrollment serum creatinine¹ (mg/dL) | 2.4 (1.7-3.3) | 1.7 (1.3-2.5) | 2.8 (2.1-3.6) | <0.001 |
| Enrollment KDIGO Stage | | | | |
| No AKI | 18 (5.4%) | 16 (12.9%) | 2 (0.9%) | |
| Stage 1 | 37 (11.0%) | 23 (18.5%) | 14 (6.6%) | <0.001 |
| Stage 2 | 169 (50.4%) | 67 (54.0%) | 102 (48.3%) | |
| Stage 3 | 111 (33.1%) | 18 (14.5%) | 93 (44.1%) | |
| Enrollment non-renal APACHE III score¹ | 54 (43-71) | 48 (37-64) | 57 (45-74) | <0.001 |

¹Median (interquartile range)
²Vasopressors and diuretics are defined as any use from 3 days before through Day 1 (day of study enrollment).
³Fluid balance is cumulative from the day prior to through the day of enrollment.

TABLE 3A
Comparison of the AUCs for Pre-Enrollment Urine
Output (UO) with Urine CCL14 Concentration
for the development of Persistent Severe AKI

| | | P-value for |
|---|---|---|
| Predictor* | ROC AUC | AUC Difference |
| uCCL14 concentration | 0.82 (0.77-0.87) | <0.0001 |
| Mean pre-enrollment weight- | 0.63 (0.57-0.70)*** | |
| adjusted hourly UO** | | |

*Two patients in the analysis cohort did not have pre-enrollment UO data and were excluded from analysis (N = 333)
**pre-enrollment includes a maximum of 24 hours of UO data
***to facilitate comparison, AUC is reported as 1 - (actual AUC) since a lower mean UO is associated with the presence of PS-AKI

TABLE 3B
Urine output and Urine CCL14 Concentration in a Logistic Regression
Model for the Development of Persistent Severe AKI

| Variable | Odds Ratio | P-value |
|---|---|---|
| Mean pre-enrollment weight- | 0.76 (0.57-1.00) | 0.053 |
| adjusted hourly UO | | |
| Urine CCL14 >1.3 and ≤13 | 5.91 (2.91-13.07) | <0.001 |
| Urine CCL14 >13 | 23.70 (9.98-61.16) | <0.001 |

TABLE 4
Operating characteristics for CCL14 concentration cutoffs from 0.2
to 30 ng/ml for the primary endpoint, persistent severe AKI.

| | | | | | Negative | Positive | Negative | Positive |
|---|---|---|---|---|---|---|---|---|
| Cutoff | % | % | | | Predictive | Predictive | Likelihood | Likelihood |
| (ng/mL) | below | above | Sensitivity | Specificity | Value | Value | Ratio | Ratio |
| 0.2 | 3.6 | 96.4 | 99% | 5% | 92% | 34% | 0.19 | 1.04 |
| | | | 95%-100% | 2%-9% | 62%-100% | 29%-39% | 0.02-1.42 | 1.01-1.08 |
| 0.3 | 6.3 | 93.7 | 99% | 9% | 95% | 35% | 0.10 | 1.09 |
| | | | 95%-100% | 6%-13% | 76%-100% | 29%-40% | 0.01-0.75 | 1.04-1.14 |
| 0.4 | 10.1 | 89.9 | 99% | 15% | 97% | 36% | 0.06 | 1.16 |
| | | | 95%-100% | 10%-20% | 85%-100% | 31%-42% | 0.01-0.45 | 1.10-1.23 |
| 0.5 | 13.4 | 86.6 | 98% | 19% | 96% | 37% | 0.10 | 1.21 |
| | | | 94%-100% | 14%-25% | 85%-99% | 32%-43% | 0.02-0.39 | 1.13-1.30 |
| 0.6 | 18.2 | 81.8 | 95% | 25% | 92% | 38% | 0.18 | 1.27 |
| | | | 90%-99% | 19%-31% | 82%-97% | 33%-44% | 0.08-0.44 | 1.17-1.38 |
| 0.7 | 21.2 | 78.8 | 94% | 28% | 90% | 39% | 0.22 | 1.31 |
| | | | 87%-97% | 23%-35% | 81%-96% | 33%-45% | 0.11-0.47 | 1.19-1.44 |
| 0.8 | 24.8 | 75.2 | 93% | 33% | 90% | 40% | 0.22 | 1.39 |
| | | | 86%-97% | 27%-40% | 82%-96% | 34%-47% | 0.11-0.44 | 1.25-1.55 |
| 0.9 | 27.5 | 72.5 | 93% | 37% | 91% | 42% | 0.19 | 1.48 |
| | | | 86%-97% | 31%-44% | 84%-96% | 36%-48% | 0.10-0.39 | 1.32-1.66 |
| 1 | 31.0 | 69.0 | 92% | 42% | 91% | 44% | 0.19 | 1.59 |
| | | | 85%-96% | 36%-49% | 84%-96% | 37%-50% | 0.10-0.37 | 1.40-1.80 |
| 1.1 | 33.7 | 66.3 | 91% | 46% | 91% | 45% | 0.20 | 1.68 |
| | | | 84%-96% | 39%-53% | 84%-96% | 38%-52% | 0.11-0.36 | 1.47-1.92 |
| 1.2 | 35.8 | 64.2 | 91% | 49% | 92% | 47% | 0.19 | 1.78 |
| | | | 84%-96% | 42%-56% | 85%-96% | 40%-53% | 0.10-0.34 | 1.55-2.05 |
| 1.3 | 37.0 | 63.0 | 91% | 51% | 92% | 47% | 0.18 | 1.84 |
| | | | 84%-96% | 44%-57% | 86%-96% | 40%-54% | 0.10-0.33 | 1.59-2.13 |
| 1.4 | 40.3 | 59.7 | 88% | 54% | 90% | 49% | 0.22 | 1.93 |
| | | | 81%-94% | 47%-61% | 84%-95% | 41%-56% | 0.13-0.37 | 1.65-2.26 |
| 1.5 | 41.2 | 58.8 | 88% | 56% | 91% | 49% | 0.21 | 1.98 |
| | | | 81%-94% | 49%-62% | 84%-95% | 42%-56% | 0.13-0.36 | 1.69-2.33 |
| 1.6 | 42.7 | 57.3 | 87% | 57% | 90% | 50% | 0.22 | 2.05 |
| | | | 80%-93% | 51%-64% | 84%-95% | 43%-57% | 0.13-0.37 | 1.73-2.42 |
| 1.7 | 43.6 | 56.4 | 86% | 58% | 90% | 50% | 0.23 | 2.07 |
| | | | 79%-92% | 51%-65% | 84%-94% | 43%-58% | 0.14-0.38 | 1.74-2.45 |
| 1.8 | 46.0 | 54.0 | 85% | 61% | 90% | 52% | 0.24 | 2.21 |
| | | | 77%-91% | 55%-68% | 84%-94% | 44%-59% | 0.15-0.38 | 1.84-2.65 |
| 1.9 | 47.5 | 52.5 | 85% | 63% | 89% | 53% | 0.24 | 2.29 |
| | | | 76%-91% | 56%-69% | 83%-94% | 45%-60% | 0.16-0.38 | 1.90-2.77 |
| 2 | 48.4 | 51.6 | 84% | 64% | 89% | 53% | 0.26 | 2.32 |
| | | | 75%-90% | 57%-70% | 83%-93% | 45%-61% | 0.17-0.39 | 1.92-2.82 |
| 2.1 | 48.4 | 51.6 | 84% | 64% | 89% | 53% | 0.26 | 2.32 |
| | | | 75%-90% | 57%-70% | 83%-93% | 45%-61% | 0.17-0.39 | 1.92-2.82 |
| 2.2 | 49.6 | 50.4 | 83% | 65% | 89% | 54% | 0.26 | 2.39 |
| | | | 74%-89% | 59%-72% | 83%-93% | 46%-62% | 0.17-0.40 | 1.96-2.91 |
| 2.3 | 50.1 | 49.9 | 83% | 66% | 89% | 54% | 0.26 | 2.45 |
| | | | 74%-89% | 60%-72% | 83%-93% | 47%-62% | 0.17-0.40 | 2.0-3.0 |
| 2.4 | 50.7 | 49.3 | 83% | 67% | 89% | 55% | 0.26 | 2.52 |
| | | | 74%-89% | 61%-73% | 83%-93% | 47%-63% | 0.17-0.39 | 2.05-3.09 |
| 2.5 | 52.2 | 47.8 | 81% | 68% | 88% | 56% | 0.28 | 2.56 |
| | | | 72%-88% | 62%-74% | 82%-92% | 48%-63% | 0.19-0.41 | 2.07-3.17 |
| 2.6 | 53.7 | 46.3 | 80% | 70% | 88% | 57% | 0.28 | 2.69 |
| | | | 71%-87% | 64%-76% | 82%-92% | 49%-65% | 0.19-0.42 | 2.15-3.35 |
| 2.7 | 54.6 | 45.4 | 80% | 72% | 88% | 58% | 0.28 | 2.81 |
| | | | 71%-87% | 65%-77% | 82%-92% | 50%-66% | 0.19-0.41 | 2.24-3.53 |
| 2.8 | 55.5 | 44.5 | 80% | 73% | 88% | 59% | 0.27 | 2.95 |
| | | | 71%-87% | 67%-79% | 83%-92% | 51%-67% | 0.19-0.40 | 2.34-3.73 |
| 2.9 | 56.1 | 43.9 | 80% | 74% | 88% | 60% | 0.27 | 3.05 |
| | | | 71%-87% | 68%-79% | 83%-93% | 51%-68% | 0.19-0.40 | 2.40-3.87 |
| 3 | 56.7 | 43.3 | 80% | 75% | 88% | 61% | 0.27 | 3.16 |
| | | | 71%-87% | 68%-80% | 83%-93% | 52%-69% | 0.18-0.39 | 2.48-4.03 |
| 3.5 | 59.7 | 40.3 | 77% | 78% | 88% | 63% | 0.29 | 3.48 |
| | | | 68%-85% | 72%-83% | 82%-92% | 54%-71% | 0.21-0.42 | 2.67-4.53 |
| 4 | 61.8 | 38.2 | 75% | 80% | 87% | 65% | 0.31 | 3.77 |
| | | | 66%-83% | 74%-85% | 82%-91% | 56%-73% | 0.22-0.43 | 2.85-5.0 |
| 4.5 | 64.5 | 35.5 | 71% | 82% | 85% | 66% | 0.36 | 3.89 |
| | | | 61%-79% | 76%-87% | 80%-90% | 56%-74% | 0.26-0.48 | 2.88-5.26 |
| 5 | 66.9 | 33. | 68% | 84% | 84% | 68% | 0.38 | 4.26 |
| | | | 59%-77% | 79%-89% | 79%-89% | 58%-76% | 0.29-0.50 | 3.08-5.90 |
| 5.5 | 69.3 | 30.7 | 65% | 86% | 83% | 69% | 0.41 | 4.54 |
| | | | 55%-73% | 81%-90% | 78%-88% | 59%-78% | 0.32-0.53 | 3.20-6.44 |
| 6 | 70.7 | 29.3 | 61% | 86% | 82% | 68% | 0.45 | 4.42 |
| | | | 51%-70% | 81%-90% | 76%-87% | 58%-77% | 0.36-0.58 | 3.09-6.33 |
| 6.5 | 71.6 | 28.4 | 58% | 86% | 81% | 67% | 0.49 | 4.22 |
| | | | 48%-68% | 81%-90% | 75%-86% | 57%-77% | 0.39-0.61 | 2.94-6.07 |
| 7 | 73.4 | 26.6 | 55% | 87% | 80% | 67% | 0.52 | 4.23 |
| | | | 45%-64% | 82%-91% | 74%-85% | 57%-77% | 0.42-0.64 | 2.89-6.19 |
| 7.5 | 75.8 | 24.2 | 49% | 88% | 78% | 67% | 0.58 | 4.09 |
| | | | 39%-59% | 83%-92% | 72%-83% | 55%-77% | 0.48-0.70 | 2.74-6.11 |
| 8 | 77.0 | 23.0 | 46% | 88% | 77% | 66% | 0.61 | 4.01 |
| | | | 37%-56% | 84%-92% | 72%-82% | 55%-77% | 0.51-0.73 | 2.65-6.07 |
| 8.5 | 77.9 | 22.1 | 46% | 90% | 77% | 69% | 0.60 | 4.54 |
| | | | 37%-56% | 85%-93% | 72%-82% | 57%-79% | 0.50-0.71 | 2.93-7.02 |
| 9 | 78.5 | 21.5 | 45% | 90% | 77% | 68% | 0.62 | 4.36 |
| | | | 35%-54% | 85%-93% | 71%-82% | 56%-79% | 0.52-0.73 | 2.81-6.76 |
| 9.5 | 78.8 | 21.2 | 45% | 90% | 77% | 69% | 0.6 | 4.56 |
| | | | 35%-54% | 86%-94% | 71%-82% | 57%-79% | 0.52-0.73 | 2.91-7.13 |
| 10 | 78.8 | 21.2 | 45% | 90% | 77% | 69% | 0.61 | 4.56 |
| | | | 35%-54% | 86%-94% | 71%-82% | 57%-79% | 0.52-0.73 | 2.91-7.13 |
| 11 | 81.2 | 18.8 | 41% | 92% | 76% | 71% | 0.64 | 5.11 |
| | | | 32%-51% | 88%-95% | 71%-81% | 59%-82% | 0.55-0.75 | 3.11-8.40 |
| 12 | 82.1 | 17.9 | 38% | 92% | 75% | 70% | 0.67 | 4.77 |
| | | | 29%-48% | 88%-95% | 70%-80% | 57%-81% | 0.58-0.78 | 2.89-7.89 |
| 13 | 83.9 | 16.1 | 35% | 93% | 75% | 72% | 0.69 | 5.32 |
| | | | 27%-45% | 89%-96% | 69%-80% | 58%-84% | 0.60-0.80 | 3.07-9.22 |
| 14 | 85.7 | 14.3 | 33% | 95% | 74% | 75% | 0.71 | 6.14 |
| | | | 24%-42% | 91%-97% | 69%-79% | 60%-86% | 0.62-0.81 | 3.33-11.3 |
| 15 | 87.2 | 12.8 | 30% | 96% | 74% | 77% | 0.73 | 6.75 |
| | | | 22%-39% | 92%-98% | 68%-79% | 61%-88% | 0.65-0.83 | 3.45-13.2 |
| 16 | 87.8 | 12.2 | 28% | 96% | 73% | 76% | 0.75 | 6.34 |
| | | | 20%-38% | 92%-98% | 68%-78% | 60%-88% | 0.67-0.85 | 3.23-12.5 |
| 17 | 87.8 | 12.2 | 28% | 96% | 73% | 76% | 0.75 | 6.34 |
| | | | 20%-38% | 92%-98% | 68%-78% | 60%-88% | 0.67-0.85 | 3.23-12.5 |
| 18 | 88.4 | 11.6 | 27% | 96% | 73% | 77% | 0.76 | 6.82 |
| | | | 19%-37% | 93%-98% | 68%-78% | 61%-89% | 0.67-0.85 | 3.35-13.9 |
| 19 | 88.7 | 11.3 | 27% | 96% | 73% | 79% | 0.75 | 7.67 |
| | | | 19%-37% | 93%-98% | 68%-78% | 63%-90% | 0.67-0.85 | 3.64-16.2 |
| 20 | 89.3 | 10.7 | 26% | 97% | 73% | 81% | 0.76 | 8.47 |
| | | | 18%-36% | 94%-99% | 67%-78% | 64%-92% | 0.68-0.85 | 3.83-18.7 |
| 21 | 89.6 | 10.4 | 25% | 97% | 73% | 80% | 0.77 | 8.18 |
| | | | 18%-35% | 94%-99% | 67%-78% | 63%-92% | 0.69-0.86 | 3.69-18.1 |
| 22 | 89.6 | 10.4 | 25% | 97% | 73% | 80% | 0.77 | 8.18 |
| | | | 18%-35% | 94%-99% | 67%-78% | 63%-92% | 0.69-0.86 | 3.69-18.1 |
| 23 | 90.1 | 9.9 | 24% | 97% | 72% | 79% | 0.79 | 7.60 |
| | | | 16%-33% | 94%-99% | 67%-77% | 61%-91% | 0.71-0.88 | 3.40-17.0 |
| 24 | 90.7 | 9.3 | 22% | 97% | 72% | 77% | 0.81 | 7.01 |
| | | | 15%-31% | 94%-99% | 66%-77% | 59%-90% | 0.73-0.89 | 3.12-15.8 |
| 25 | 91.0 | 9.0 | 21% | 97% | 71% | 77% | 0.82 | 6.72 |
| | | | 14%-30% | 94%-99% | 66%-76% | 58%-90% | 0.74-0.90 | 2.98-15.2 |
| 26 | 91.3 | 8.7 | 20% | 97% | 71% | 76% | 0.83 | 6.43 |
| | | | 13%-29% | 94%-99% | 66%-76% | 56%-90% | 0.75-0.91 | 2.83-14.6 |
| 27 | 91.6 | 8.4 | 19% | 97% | 71% | 75% | 0.84 | 6.14 |
| | | | 12%-28% | 94%-99% | 66%-76% | 55%-89% | 0.76-0.92 | 2.69-14.0 |
| 28 | 91.6 | 8.4 | 19% | 97% | 71% | 75% | 0.84 | 6.14 |
| | | | 12%-28% | 94%-99% | 66%-76% | 55%-89% | 0.76-0.92 | 2.69-14.0 |
| 29 | 91.6 | 8.4 | 19% | 97% | 71% | 75% | 0.84 | 6.14 |
| | | | 12%-28% | 94%-99% | 66%-76% | 55%-89% | 0.76-0.92 | 2.69-14.0 |
| 30 | 100.0 | 0.0 | 0% | 100% | 67% | na | 1.0 | na |
| | | | 0%-0% | 100%-100% | 62%-72% | | 1.0-1.0 | |

TABLE 5
Time to Secondary Outcomes (RRT and Death)

| Secondary Endpoint | Median (IQR) in days from enrollment |
|---|---|
| RRT | 2.8 (1.7-7.2) |
| Death | 15.5 (5.4-30.1) |
| RRT or death | 4.3 (2.0-14.0) |

TABLE 6
Multivariable logistic regression model using clinical variables
for prediction of persistent severe AKI without (Reference Model)
and with (New Model) urinary CCL14 as a categorical variable with
three levels stratified by two cutoffs at 1.3 and 13 ng/mL.

| | Reference Model | | New Model with Urine CCL14 | |
|---|---|---|---|---|
| Variable | Odds Ratio | P-value | Odds Ratio | P-value |
| Body mass index | 0.79 (0.54-1.13) | 0.209 | 0.81 (0.54-1.18) | 0.292 |
| Non-renal APACHE III score | 1.47 (1.10-2.00) | 0.011 | 1.38 (1.01-1.92) | 0.047 |
| Serum creatinine trajectory† | 1.54 (1.15-2.11) | 0.005 | 1.47 (1.07-2.05) | 0.020 |
| KDIGO stage at enrollment | 5.43 (3.56-8.62) | <0.001 | 4.45 (2.86-7.20) | <0.001 |
| Diabetes | 0.56 (0.29-1.09) | 0.091 | 0.50 (0.24-1.01) | 0.055 |
| Urine CCL14 >1.3 and ≤13 | Not Included | NA | 3.82 (1.73-9.12) | 0.001 |
| Urine CCL14 >13 | Not Included | NA | 10.4 (3.89-29.9) | <0.001 |

†Change in serum creatinine concentration over the prior day as determined using two serum creatinine results with mean (±SD) collection times at 18 (±9) h and 7 (±4) h prior to enrollment

For the categorical CCL14 variable, CCL14≤1.3 ng/ml was the reference level All numeric variables were standardized by subtracting the mean and dividing by the standard deviation; N=312 (34% Persistent). The clinical variables in the reference model were selected based on association with persistent severe AKI as described previously.9

TABLE 7
NRI and IDI analysis of the addition of urinary CCL14 stratified by
two cutoffs at 1.3 and 13 ng/mL to the clinical model in Table 3.

| | Value | 95% CI | P-value |
|---|---|---|---|
| IDI | 0.06 | 0.03-0.09 | <0.001 |
| IDI_event | 0.04 | 0.02-0.07 | 0.001 |
| IDI_non_event | 0.02 | 0.01-0.04 | 0.003 |
| cfNRI | 0.53 | 0.30-0.77 | <0.001 |
| cfNRI_event | 0.39 | 0.20-0.58 | <0.001 |
| cfNRI_non_event | 0.14 | 0.00-0.28 | 0.044 |
| AUC_ref_model | 0.86 | 0.82-0.90 | <0.001 |
| AUC_new_model | 0.88 | 0.85-0.92 | <0.001 |
| AUC difference | 0.03 | 0.01-0.05 | 0.018 |

event = persistent severe AKI; non_event = not persistent severe AKI.

Clinical variables in the reference model are body mass index, non-renal APACHE III score, serum creatinine trajectory, KDIGO stage at enrollment, and diabetes.

TABLE 8
Multivariable logistic regression model using clinical variables for prediction of
persistent severe AKI without (Reference Model) and with (New Model) urinary CCL14
as a categorical variable with three levels stratified by two cutoffs at 1.3 and
13 ng/mL. and excluding those found to not have Stage 2 or 3 AKI at enrollment.

| | Reference Model | | New Model with Urine CCL14 | |
|---|---|---|---|---|
| Variable | Odds Ratio | P-value | Odds Ratio | P-value |
| Body mass index | 0.78 (0.53-1.12) | 0.194 | 0.81 (0.54-1.17) | 0.281 |
| Non-renal APACHE III score | 1.47 (1.10-2.00) | 0.011 | 1.37 (1.00-1.90) | 0.052 |
| Serum creatinine trajectory† | 1.52 (1.14-2.09) | 0.006 | 1.44 (1.06-2.01) | 0.023 |
| KDIGO stage at enrollment | 7.02 (3.92-12.96) | <0.001 | 5.32 (2.86-10.2) | <0.001 |
| Diabetes | 0.58 (0.29-1.11) | 0.105 | 0.50 (0.24-1.02) | 0.058 |
| Urine CCL14 >1.3 and ≤13 | Not Included | NA | 3.77 (1.71-8.97) | 0.002 |
| Urine CCL14 >13 | Not Included | NA | 10.6 (3.96-30.8) | <0.001 |

†Change in serum creatinine concentration over the prior day as determined using two serum creatinine results with mean (±SD) collection times at 19 (±9) h and 7 (±4) h prior to enrollment

For the categorical CCL14 variable, CCL14≤1.3 ng/mL was the reference level

All numeric variables were standardized by subtracting the mean and dividing by the standard deviation; N=260 (40% Persistent). The clinical variables in the reference model were selected based on association with persistent severe AKI as described previously.9

### Example 2

### Methods

### Research Background

We conducted a pre-planned analysis of serial measurement of urinary CCL14 early after development of moderate to severe AKI in a mixed population of critically ill adults. The analysis cohort is composed of pooled cohorts of patients from the Ruby and Sapphire studies.

### Subjects

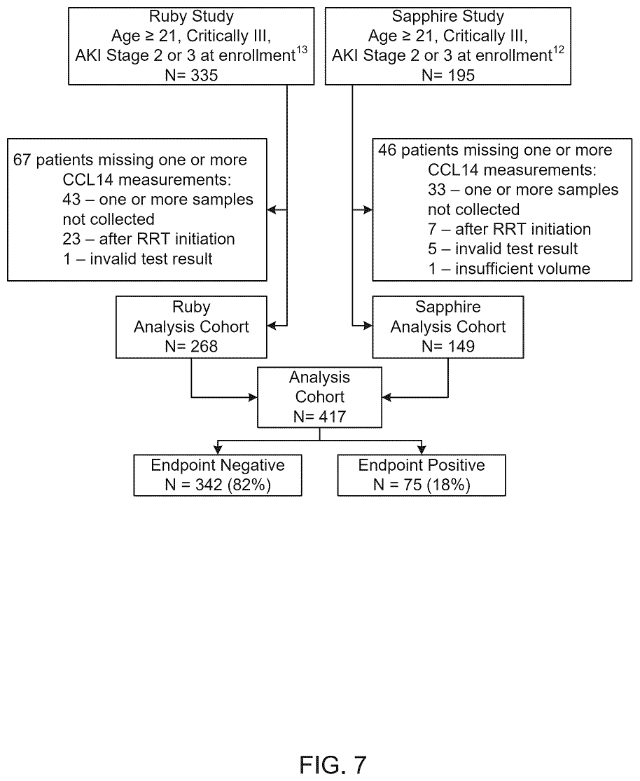

Ruby enrolled subjects as described in Example 1 herein. Sapphire enrolled adult critically ill patients with cardiac or respiratory dysfunction without known stage 2-3 AKI at the time of enrolment from 35 sites in Europe and North America (summarized in FIG. 1). (Kashani K, et al., Critical Care, 2013; 17 (R25): 1-12). Samples from a subset of Sapphire patients who developed stage 2-3 AKI within one-week of enrolment were included in the present analysis. For the purposes of this analysis, in the Sapphire cohort serial biomarker measurements were included from the point at which patients met stage 2-3 AKI criteria, mirroring the time-course of enrolment and biomarker measurement in the RUBY study. For both studies, patients without CCL14 concentrations for all 3 samples or who received RRT or died before the 3^{rd} sample were excluded from the primary analysis. Patients were recruited on the basis of AKI assessment using creatinine and urine output criteria by local site investigators and all patients enrolled were included on an intention to diagnose basis.

### Sample Collection and Testing

In the Ruby study, urine samples were collected twice daily for 3 days from enrolment and then once daily for 4 days. In the Sapphire study, urine samples were collected twice daily for 4 days from enrolment and then once daily for 3 days. In both studies, urine samples were centrifuged, flash frozen, stored at or below −70° C., and thawed prior to sample testing. The primary analysis utilized CCL14 concentrations from the first 3 scheduled urine collections in Ruby and the first 3 urine collections after onset of KDIGO stage 2 or 3 AKI in Sapphire for each patient. Technicians who were blinded to the clinical data measured CCL14 concentrations in the samples using the NEPHROCLEAR™ CCL14 Test on the Astute 140® Meter (Astute Medical Inc., San Diego, CA).

### Clinical Endpoint

The primary endpoint for the present analysis was the development of persistent severe AKI, as described previously. In brief, patients who developed 72 consecutive hours of stage 3 AKI, commencing within 48 h of first sample collection, or those who died following stage 3 AKI or received RRT prior to 48 h from first sample collection or within 72 consecutive hours of stage 3 AKI, were considered endpoint positive. For the purposes of determining the primary endpoint, each patient's baseline serum creatinine was determined as described in the relevant studies.

### Research Question

We hypothesized that, as biomarker of underlying renal inflammation during tissue injury and repair, urinary CCL14 be informative about clinical course of AKI. We therefore sought to characterize the distribution of CCL14 values over time and the clinical correlations of various patterns.

### Statistical Analysis

We stratified the CCL14 concentrations in the first 3 urine collections into three levels: Low (≤1.3 ng/ml), Medium (>1.3 to ≤13 ng/mL), and High (>13 ng/ml), using previously determined clinical-risk cut-offs (Koyner, J. L, Kidney360. 2022 Mar. 24;3 (7): 1158-1168). The high sensitivity cut-off at 1.3 ng/ml CCL14 was derived to identify the majority of patients at risk of persistent severe AKI and the high specificity cut-off at 13 ng/ml to identify the highest risk patients where PS-AKI is most likely. To assess the validity of using a pooled dataset we used the Cochran Q test to assess heterogeneity of relative (RR) risk between individual time points across Ruby and Sapphire. Patients in the pooled analysis cohort were then grouped by the pattern of CCL14 levels across the 3 samples. Trajectories of CCL14 level where then compared based on proportion of patients positive for persistent severe AKI within each group. Continuous and categorical variables were analysed using the Wilcoxon Rank Sum and Fisher's exact tests, respectively. Two-sided p-values <0.05 were considered statistically significant. All analyses were performed using R 4.0.2 (R Foundation for Statistical Computing. Vienna, Austria).

### Sensitivity Analyses

Our primary analysis is based on those with a full set of 3 CCL14 measurements over 24 h to allow assessment of a true trajectory, rather than point measurement and because missing CCL14 values might not be missing a random. However, we also performed a sensitivity analysis including all individuals with one or more CCL14 measurements after enrolment to assess consistency of our findings in this population. In a second sensitivity analysis, we used alternative pairs of cutoff values with sensitivities and specificities different from those at 1.3 and 13 ng/ml to explore potential differences in our main conclusions.

### Extended Trajectory of CCL-14

CCL14 measurement in urine samples collected at least daily allowed assessment of change in CCL14 trajectory over a longer time and it's relationship to outcome on day 7. We performed rolling assessment of the PS-AKI composite endpoint over time-that is at each timepoint of measurement the occurrence of 72 h of sustained stage 3 AKI commencing within 48 h or death or RRT with 48 h of that measurement was assessed. Ability of urinary CCL14 at any timepoint (as a continuous variable) to discriminate the rolling endpoint was assessed as area under the Receiver-Operative Curve (ROC-AUC) with 95% confidence determined by the DeLong method. In the sensitivity analysis including patients with missing CCL14 values, the association between change in CCL14 stratified by baseline was essentially unchanged apart from overall worse outcomes for ‘unchanged’ values reflecting inclusion of patients where measurement was truncated by death or need for RRT (FIG. 9), with a similar pattern of association in the logistic regression (Table 12).

### Results

### Patients

The Ruby study recruited 364 patients within 36 h of diagnosis of stage 2-3 while of the 723 patients critically ill patients without AKI enrolled in Sapphire, 212 developed stage 2-3 AKI and were eligible for inclusion in this pooled analysis. After further exclusion of patients without 3 consecutive measurements of urinary CCL14 after meeting enrolment criteria, a total of 417, 268 patients from the Ruby study and 149 patients from Sapphire were included in our primary analysis (FIG. 1). In addition, a further 111 patients had one or two valid urinary measurements within 36 h of enrolment and were included in a sensitivity analysis of 528 patients. Of the pooled population 75 (18%) reached the primary endpoint of PS-AKI (72 hrs of sustained stage 3 AKI or RRT or death prior to recovery of kidney function), while in the sensitivity analysis 135 (26%) reached the endpoint, often reflecting the effect of early death or anuria preventing collection of a full set of study samples. In the Sapphire cohort only 7.4% of patients reached the primary endpoint compared to 24% on Ruby (Table 13), reflecting lower overall severity of AKI in the Sapphire cohort, AKI stage 3 at time of first sample collection 4% vs 30%. Conversely, the Sapphire cohort had higher non-renal APACHE-2 score (62 vs. 52) and use of mechanical ventilation (85 vs 54%) In the pooled primary dataset median age was 65 years and 59% were male (Table 13). Baseline kidney function, demographics, illness severity and organ support did not significantly differ between patients with PS-AKI endpoint than those without, however fluid balance on the first day after enrolment was significantly more positive in those who developed PS-AKI, (2.3L vs. 3.4L, p=0.04). Overall, despite the differences between the studies, the relative risks analysis showed that they behave similarly across the CCL14 strata at each time point justifying the pooled cohort analysis (Table 9)

### Ccl14 Trajectory Over the First Day and the Primary Composite Endpoint

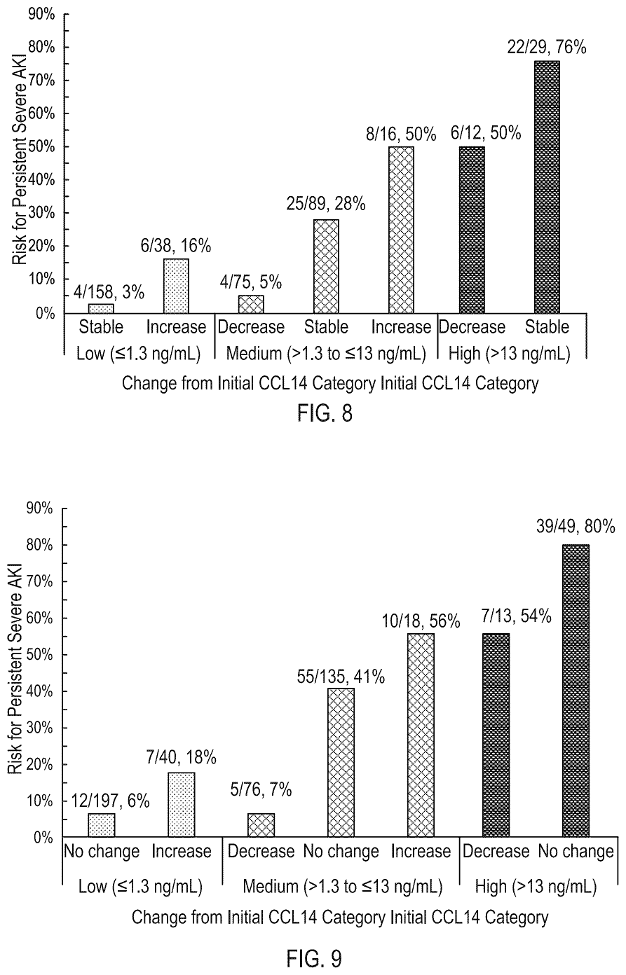

Initial CCL14 levels were low (≤1.3 ng/ml) in 196 (47%), medium (>1.3 to ≤13 ng/ml) in 180 (43%) and high (>13 ng/ml) in 41 (9.8%) (Table 14). The initial CCL14 category strongly correlated with the primary endpoint. Importantly the association between CCL14 category and PS-AKI outcome was essentially unchanged at the 12 h and 24 h timepoints. In line with this observation, in the majority of cases (66%) CCL14 category was unchanged from 0 to 24 h timepoints and in only one patient did a 2-level change occur (FIG. 8, Table 10). The graph of FIG. 8 depicts the change from initial CCL14 category and the risk for persistent severe AKI. Where changes occurred, they were generally consistent in direction with only 24/417 (6%) showing a fluctuating course in CCL14 category (Table 10). In patients experiencing a change in CCL14 category we classified patients as decreasing or increasing CCL14 category between 0 and 24 h and stratified these by the initial CCL14 levels. Across all initial categories a change in CCL14 category was associated with a corresponding change in risk of PS-AKI (FIG. 8). We assessed the additional information provided by changes in CCL14 level, accounting for the initial value in multivariable logistic regression. Accounting for initial CCL14 category, a decrease in CCL14 category in the first 24 h was associated with decreased risk of persistent severe AKI, odds ratio (OR) 0.2 (95% CI 0.08-0.45, p<0.001). An increase in category was associated with increased risk, OR 4.04 (1.75-9.46, p=0.001). When tested there was no significant interaction between initial status and change (p=0.36 for interaction term)-Table 15.

### Sensitivity Analyses

In the sensitivity analysis including patients with missing CCL14 values, association between change in CCL14 stratified by baseline was essentially unchanged apart from overall worse outcomes for ‘unchanged’ values reflecting inclusion of patients where measurement was truncated by death or need for RRT (FIG. 9), with a similar pattern of association in the logistic regression (Table 12).

In the second sensitivity analysis using alternative cutoff values, 2 additional cutoffs above and below 1.3 and 13 were considered, giving a total of 6 cutoffs and 9 different combinations of low and high cutoffs (Table 16). The additional cutoff values, 0.8, 1.8, 9, and 17 were selected based on their sensitivities and specificities relative to the original, predetermined cutoffs, 1.3 and 13 ng/ml (Example 1 Table 4). The different combinations of cutoffs yielded CCL14 categories that are strongly correlated with the persistent severe AKI endpoint, similar to that observed with the original cutoffs of 1.3 and 13 ng/ml (compare Tables 14 and 17). In addition, the odds ratios in a multivariable logistic regression of the PS-AKI endpoint display a similar pattern where the initial and change in CCL14 category are both statistically significant independent predictors of the endpoint across all the combinations of cutoffs (compare Tables 15 and 18). Finally, the trend of increasing risk for the primary endpoint with an increasing change in CCL14 categories within an initial CCL14 category can be observed across all combinations of low and high cutoff values (compare FIG. 8 and Table 19).

### Extended CCL14 Trajectory

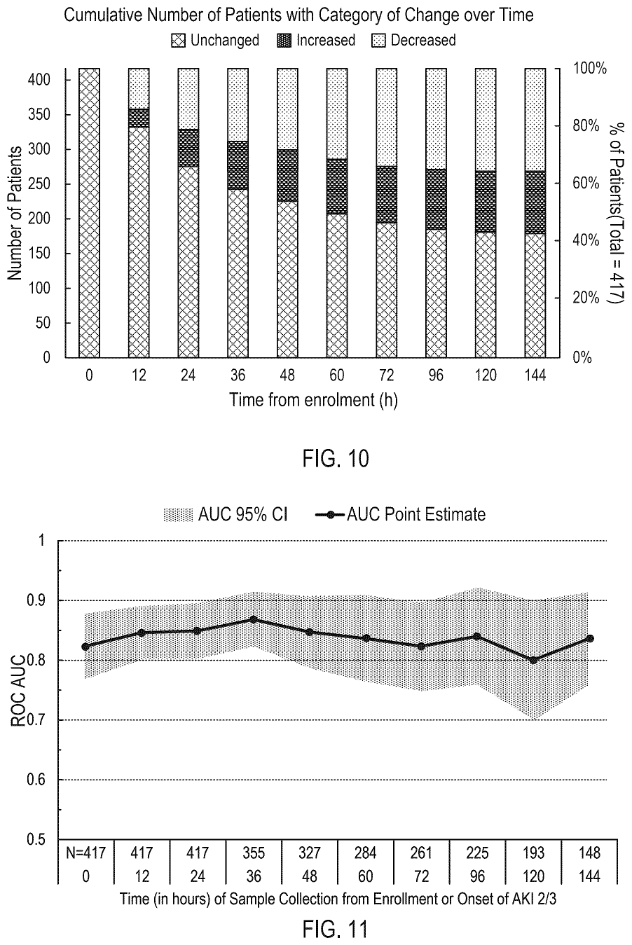

Urinary CCL14 measurements were conducted at least daily up to 7 days after enrolment in surviving patients remaining in ICU with urinary catheter and without RRT. At 36 h, 355 patients of the original 417 had measurements available which progressively declined to 148 by 144 h. Compared to the original value, over time, CCL14 values tended to decrease more than increase (FIG. 10) and 67% of patients had a low level of CCL14 as their last measurement. Referring to the graph of FIG. 11, the ROC-AUC shown for each timepoint with 95% CI for CCL14 over study period. Endpoint assessment was based on clinical data collected through up to 7 days, and last observation carried forward was used to impute right-censored data. Rolling assessment of ability to predict the PS-AKI composite endpoint commencing within 48 h demonstrated sustained ability to well-discriminate the endpoint with ROCAUC >0.8 through to the 144 h timepoint (FIG. 11).

### DISCUSSION

We demonstrate important clinical features of the CCL14 assay that will both guide clinicians in interpretation of this assay and add additional confidence to its interpretation in real-world clinical settings. Importantly we demonstrate that in two-thirds of patients with moderate to severe AKI, CCL14 levels remained stable over a 24 h period, and where changes occurred this indicated a corresponding change in risk level for persistent severe AKI. There are two important clinical implications of these findings. First, even if measurement of CCL14 is delayed after first diagnosis of stage 2-3 AKI the result is likely to provide at least as good prognostication of clinical course of AKI as that provided by the original clinical trials that derived and validate the assay. In other words, CCL14 does not represent a transient damage or stress signal that falls quickly during severe injury but is an indication of newly arisen but persistent biological process within a severely injured kidney. Second, the serial measurement of CCL14 may provide complementary clinical information that modifies assessment of risk over time by reflecting potentially modifiable pathology leading to a changing prognosis over time. In particular, for patients with intermediate CCL14 measurement, a fall into the low category is associated with a step down in risk to be similar to that of an initially low measurement, while conversely, an increase to high is associated with a large step up in risk. Notably, although CCL14 levels overall were generally stable, 45% of those with an initial medium value experienced a shift in category within the first 24 h. Thus, for patients with an initial measurement in the median risk category, serial measurement provides opportunity to update risk assessment with relatively frequent and clinically important changes possible. Conversely, for patients with an initial CCL14 in the high-risk category, a change to a lower category, while being associated with a clear reduction in risk, was still associated with a >50% incidence of the primary endpoint suggesting little value in ‘watching and waiting’ in the context of a high category result. Finally, it appears that CCL14 retains ability to discriminate the composite endpoint over 6 days after initial moderate to severe AKI diagnosis, suggesting CCL14 is potentially, a flexible tool that could be used to assess underlying kidney pathophysiology through the time-course of AKI, which extends over 1 week from initial diagnosis. This work therefore represents an important contribution to defining the clinical utility and implementation of the CCL14 assay for the identification of those at risk for persistent severe AKI, giving clinicians confidence in its interpretation across the AKI time-course.

Persistent severe AKI is a critical clinical event, as these patients are at highest risk for the most adverse cardiovascular and kidney outcomes, while transient, self-limiting AKI has a much more (though not completely) benign prognosis. Several distinct recovery phenotypes have been identified following an episode of stage 2 or 3 AKI; however, those with relapsing and/or unrecovered AKI have highest risk for 1-year mortality (up to 45%). Similarly, amongst patients with septic shock, AKI persisting for >48 h and unrecovered at discharge, has been associated with an over 5-fold risk of death by one year after adjustment for differences in illness severity and demographics. The same association has also been recently observed in the context of COVID-19 associated AKI, where persistent AKI at one week was associated with highest odds ratio for death of 7.6 compared to no AKI. CCL14 fills a diagnostic gap by providing important information regarding the clinical course of AKI which is in turn a critical factor in determining overall clinical outcomes. Importantly this information cannot easily be derived from existing AKI criteria nor by biomarkers directed at the initiation of development of AKI rather than the progression of kidney tubular injury in AKI.

The ability to categorise patients as a high or low risk of persistent severe AKI, RRT or death has the potential to significantly refine patient care. This includes important aspects of routine care such as avoidance of nephrotoxic medication and assessment of dosing of renally-excreted drugs. Furthermore, application of guideline-based AKI care is often hampered by insufficient prognostic information and thus in practice is often not delivered. For instance intervention appropriate for early AKI, for example, aggressive hemodynamic resuscitation, may be futile or even counter-productive in the setting of established AKI leading to clinical uncertainty about therapy choice. Such uncertainty regarding risk-benefit of interventions may explain the failure of many interventional trials in AKI to improve outcomes. A key feature of any successful interventional study in the ICU is a pragmatic design ensuring any intervention can be applied to an easily identified sub-population of patients likely to benefit in real-world clinical conditions. In this context the ability of CCL14 to provide consistent clinical information over time will greatly facilitate trial design and inclusion. Similarly, when used as a clinical tool CCL14 can be similarly applied to a wide range of patients across the time-course of AKI. Such use may include prognostication of need for RRT in the ICU, a setting where conventional AKI diagnostic criteria and early AKI biomarkers have failed to discriminate patients who will benefit from pre-emptive RRT-initiation.

In conclusion, we have demonstrated the consistency of CCL14 for the identification of patients at high risk for the development of severe persistent AKI across serial sampling and shown where changes occur these are paralleled in clinical outcomes. CCL14 is therefore likely to be a useful clinical tool that can be flexibly implemented in the prognostication of patients with moderate to severe AKI in real world conditions and potentially monitored over time. These results have immediate utility in helping to guide patient care and may facilitate future pragmatic clinical trials.

TABLE 9
Sapphire and Ruby Poolability Analysis Cochran Q test to assess heterogeneity of relative
(RR) risk for PS-AKI between individual time points across Ruby and Sapphire.

| | | | | | | | | | | Combined 1^{st}, 2^{nd} and 3^{rd} | | |
|---|---|---|---|---|---|---|---|---|---|---|---|---|
| | 1^{st} Time Point | | | 2^{nd} Time Point | | | 3^{rd} Time Point | | | Time Points | | |
| | RR in | RR in | | RR in | RR in | | RR in | RR in | | RR in | RR in | |
| Stratum | Ruby | Sapphire | p-value | Ruby | Sapphire | p-value | Ruby | Sapphire | p-value | Ruby | Sapphire | p-value |
| M to L | 3.5 | 3.3 | 0.948 | 6.8 | 17 | 0.429 | 4.2 | 15 | 0.252 | 13 | 18 | 0.710 |
| | (1.7-7.3) | (0.62-17) | | (3.0-15) | (2.1-134) | | (2.1-8.5) | (1.9-122) | | (6.5-27) | (4.1-77) | |
| H to L | 8.8 | 37 | 0.070 | 14 | 46 | 0.307 | 10 | 51 | 0.152 | 31 | 83 | 0.219 |
| | (4.3-18) | (9.0-153) | | (6.3-32) | (5.7-367) | | (5.3-20) | (6.4-400) | | (15-62) | (20-345) | |

TABLE 10
Pattern of CCL14 category across the 3 time points and
risk for persistent severe AKI for each pattern.

| 1^{st} Time | 2^{nd} Time | 3^{rd} Time | | | Risk for |
|---|---|---|---|---|---|
| Point | Point | Point | N | % Total | endpoint |
| L | L | L | 158 | 38% | 3% |
| L | L | M | 19 | 5% | 11% |
| L | M | L | 8 | 2% | 25% |
| L | M | M | 10 | 2% | 20% |
| L | H | M | 1 | <1% | 0% |
| M | L | L | 40 | 10% | 3% |
| M | L | M | 11 | 3% | 0% |
| M | M | L | 24 | 6% | 13% |
| M | M | M | 89 | 21% | 28% |
| M | M | H | 9 | 2% | 67% |
| M | H | M | 3 | 1% | 33% |
| M | H | H | 4 | 1% | 25% |
| H | M | L | 1 | <1% | 0% |
| H | M | M | 6 | 1% | 67% |
| H | M | H | 1 | <1% | 0% |
| H | H | M | 4 | 1% | 50% |
| H | H | H | 29 | 7% | 76% |

| | | | |
|---|---|---|---|
| Total | 417 | 100% | NA |

“L” = CCL14 concentration ≤1.3 ng/mL.
“M” = CCL14 concentration >1.3 and ≤13 ng/mL.
“H” = CCL14 concentration >13 ng/mL.
Patients with 3 time points in Ruby and Sapphire (analysis cohort).

TABLE 11
Multivariable logistic regression model for PS-AKI Endpoint
with initial CCL14 values and percentage change in concentration
of the 3 CCL14 values (slope) over the first day as predictor
variables. - Complete case cohort.

| | Odds | | |
|---|---|---|---|
| Variable | Ratio | 95% CI | p-value |
| Log Initial CCL14 concentration (ng/ml) | 4.78 | 3.40-6.94 | <0.001 |
| Transformed percentage change in | 1.75 | 1.35-2.29 | <0.001 |
| concentration (% change per hr) | | | |

Interaction term was eliminated from the model as there was no statistically significant interaction between initial CCL14 value and percentage change in concentration (slope). Initial CCL14 value was log-transformed. Percentage change in concentration was transformed using the Box-Cox method with 1=−2.

Both the initial CCL14 concentration and the percentage change in concentration in the new model are statistically significant (p<0.001). In addition, both models offer nearly the same discriminatory performance for the endpoint as measured by the ROC AUC: 0.849 for the model with categorical CCL14 variables vs. 0.852 for the model with continuous CCL14 variables (p=0.78 for the AUC difference). These results indicate that both initial CCL14 level and change in CCL14 level are significant predictors of persistent severe AKI both when CCL14 is categorized using cut-offs and when CCL14 is treated as a continuous variable.

Distribution of percentage change in the concentration of the 3 CCL14 values (linear slope) stratified by the initial CCL14 category: +*

TABLE 12
Multivariable Logistic regression model for PS-AKI Endpoint
with initial CCL14 category and change in CCL14 category
over the first day as predictor variables. - sensitivity
analysis with patients with missing CCL-14 values included

| Variable | Odds Ratio | 95% CI | p-value |
|---|---|---|---|
| Initial Category: M | 9.64 | 5.43-18.0 | <0.001 |
| Initial Category: H | 72.7 | 32.5-175 | <0.001 |
| Change in Category: Decrease | 0.15 | 0.07-0.30 | <0.001 |
| Change in Category: Increase | 2.57 | 1.23-5.34 | 0.001 |

Reference levels: Initial Category: L and Change in Category: Stable. Interaction term was eliminated from the model as there was no statistically significant interaction between initial category and change in category.

TABLE 13
Baseline characteristics of analysis cohort. Study endpoint was
development of persistent severe AKI lasting at least 72 hours

| | | PS-AKI | PS-AKI | |
|---|---|---|---|---|
| | | endpoint | Endpoint | p |
| | All patients | negative | positive | value |
| Patients | 417 | 342 | 75 | |
| Male | 248 (59%) | 201 (59%) | 47 (63%) | 0.604 |
| Age¹ (years) | 65 (55-73) | 65 (55-74) | 64 (56-69) | 0.107 |
| BMI¹ (kg/m²) | 30 (26-36) | 31 (26-37) | 28 (25-34) | 0.138 |
| Race | | | | |
| Black or African American | 50 (12%) | 43 (13%) | 7 (9%) | 0.615 |
| Other/Unknown | 31 (7%) | 24 (7%) | 7 (9%) | |
| White or Caucasian | 336 (81%) | 275 (80%) | 61 (81%) | |
| Chronic comorbidities | | | | |
| Chronic kidney disease | 58 (14%) | 42 (12%) | 16 (21%) | 0.063 |
| Diabetes mellitus | 155 (37%) | 128 (37%) | 27 (36%) | 0.895 |
| Heart failure | 99 (24%) | 82 (24%) | 17 (23%) | 0.882 |
| Coronary artery disease | 144 (35%) | 121 (35%) | 23 (31%) | 0.503 |
| Hypertension | 289 (69%) | 239 (70%) | 50 (67%) | 0.583 |
| COPD | 81 (19%) | 66 (19%) | 15 (20%) | 0.873 |
| Cancer | 108 (26%) | 89 (26%) | 19 (25%) | 1.000 |
| Reason for ICU admission | | | | |
| Respiratory | 150 (36%) | 121 (35%) | 29 (39%) | 0.597 |
| Surgery | 136 (33%) | 117 (34%) | 19 (25%) | 0.173 |
| Cardiovascular | 176 (42%) | 136 (40%) | 40 (53%) | 0.039 |
| Sepsis | 98 (24%) | 77 (23%) | 21 (28%) | 0.367 |
| Neurological | 27 (6%) | 24 (7%) | 3 (4%) | 0.443 |
| Trauma | 11 (3%) | 11 (3%) | 0 (0%) | 0.226 |
| Other | 107 (26%) | 89 (26%) | 18 (24%) | 0.772 |
| Baseline serum creatinine¹ (mg/dL) | 1.0 (0.7-1.2) | 1.0 (0.7-1.2) | 1.0 (0.8-1.4) | 0.137 |
| Baseline eGFR | 76 (54-95) | 76 (55-95) | 76 (53-96) | 0.319 |
| Non-renal APACHE III score at enrollment | 55 (43-74) | 56 (42-73) | 55 (45-79) | 0.379 |
| Time difference (in hours) between | 11 (9-14) | 11 (9-14) | 10 (6-13) | 0.011 |
| 2^{nd} and 1^{st} samples¹ | | | | |
| Time difference (in hours) between | 24 (21-26) | 24 (22-26) | 22 (19-25) | 0.005 |
| 3^{rd} and 1^{st} samples¹ | | | | |
| Time difference (in hours) between | 12 (11-13) | 12 (11-13) | 12 (11-13) | 0.564 |
| 3^{rd} and 2^{st} samples¹ | | | | |
| Fluid balance¹ (mL) on Day 1 | 2426 (745-5067) | 2300 (623-4799) | 3373 (1219-6552) | 0.040 |
| Diuretics use on Day 1 | 162 (39%) | 131 (38%) | 31 (41%) | 0.695 |
| Vasopressor use on Day 1 | 199 (48%) | 165 (48%) | 34 (45%) | 0.702 |
| Mechanical ventilation use on Day 1 | 273 (65%) | 228 (67%) | 45 (60%) | 0.285 |
| KDIGO Stage at 1^{st} Sample | | | | |
| Collection* | | | | |
| No AKI | 51 (12%) | 51 (15%) | 0 (0%) | <0.001 |
| Stage 1 | 49 (12%) | 49 (14%) | 0 (0%) | |
| Stage 2 | 231 (55%) | 204 (60%) | 27 (36%) | |
| Stage 3 | 86 (21%) | 38 (11%) | 48 (64%) | |
| RRT within 7 days | 43 (10%) | 14 (4%) | 29 (39%) | <0.001 |
| Death within 7 days | 29 (7%) | 19 (6%) | 10 (13%) | 0.024 |
| RRT or death within 7 days | 69 (17%) | 31 (9%) | 38 (51%) | <0.001 |

¹Median (interquartile range)
*KDIGO stage after retrospective re-adjudication of AKI status

TABLE 14
CCL14 concentrations at three time points in the first 24 h, categorised
by pre-defined CCL-14 cut-off and the study endpoint.

| | | PS-AKI Endpoint | PS-AKI Endpoint | |
|---|---|---|---|---|
| | All patients | Negative | Positive | p-value |
| 1^{st} Sample | | | | |
| Median of all results (IQR) | 1.44 (0.60-4.53) | 1.12 (0.52-2.79) | 7.29 (4.03-24.49) | <0.001 |
| ≤1.3 ng/mL | 196 (47%) | 186 (54%) | 10 (13%) | <0.001 |
| >1.3 and ≤13 ng/mL | 180 (43%) | 143 (42%) | 37 (49%) | |
| >13 ng/mL | 41 (10%) | 13 (4%) | 28 (37%) | |
| 2^{nd} Sample | | | | |
| Median of all results (IQR) | 1.05 (0.47-3.94) | 0.82 (0.40-2.30) | 5.97 (3.10-21.38) | <0.001 |
| ≤1.3 ng/mL | 228 (55%) | 221 (65%) | 7 (9%) | <0.001 |
| >1.3 and ≤13 ng/mL | 148 (35%) | 106 (31%) | 42 (56%) | |
| >13 ng/mL | 41 (10%) | 15 (4%) | 26 (35%) | |
| 3^{rd} Sample | | | | |
| Median of all results (IQR) | 0.99 (0.44-3.12) | 0.78 (0.38-1.82) | 7.85 (2.31-19.64) | <0.001 |
| ≤1.3 ng/mL | 231 (55%) | 221 (65%) | 10 (13%) | <0.001 |
| >1.3 and ≤13 ng/mL | 143 (34%) | 107 (31%) | 36 (48%) | |
| >13 ng/mL | 43 (10%) | 14 (4%) | 29 (39%) | |

TABLE 15
Multivariable logistic regression model for PS-AKI Endpoint with
initial CCL14 category and change in CCL14 category over the
first day as predictor variables. - primary analysis cohort.

| Variable | Odds Ratio | 95% CI | p-value |
|---|---|---|---|
| Initial Category: M | 9.91 | 4.64-23.3 | <0.001 |
| Initial Category: H | 107 | 39.0-326 | <0.001 |
| Change in Category: Decrease | 0.20 | 0.08-0.45 | <0.001 |
| Change in Category: Increase | 4.04 | 1.75-9.46 | 0.001 |

Reference levels: Initial Category: L and Change in Category: Stable.

Interaction term was eliminated from the model as there was no statistically significant interaction between initial category and change in category.

TABLE 16
Nine different combinations of 3 low and 3 high cutoff values.

| Low cutoff (ng/mL) | High cutoff (ng/mL) |
|---|---|
| 0.8 | 9 |
| 1.3 | 9 |
| 1.8 | 9 |
| 0.8 | 13 |
| 1.3 | 13 |
| 1.8 | 13 |
| 0.8 | 17 |
| 1.3 | 17 |
| 1.8 | 17 |

TABLE 17
CCL14 concentrations at three time points in the first 24 h, categorized by
9 different combinations of low and high cutoffs and the study endpoint.

| Low | High | | PS-AKI | PS-AKI | |
|---|---|---|---|---|---|
| cutoff | cutoff | | Endpoint | Endpoint | |
| (ng/mL) | (ng/ml) | All patients | Negative | Positive | p-value |

1^{st} Sample

| NA | NA | Median of all | 1.44 | | 1.12 | | 7.29 | | <0.001 |
|---|---|---|---|---|---|---|---|---|---|
| | | results (IQR) | (0.60-4.53) | | (0.52-2.79) | | (4.03-24.49) | | |
| 0.8 | 9 | ≤ Low cutoff | 138 | (33%) | 131 | (38%) | 7 | (9%) | <0.001 |
| | | > Low and ≤ High | 222 | (53%) | 185 | (54%) | 37 | (49%) | |
| | | > High cutoff | 57 | (14%) | 26 | (8%) | 31 | (41%) | |
| 0.8 | 13 | ≤ Low cutoff | 138 | (33%) | 131 | (38%) | 7 | (9%) | <0.001 |
| | | > Low and ≤ High | 238 | (57%) | 198 | (58%) | 40 | (53%) | |
| | | > High cutoff | 41 | (10%) | 13 | (4%) | 28 | (37%) | |
| 0.8 | 17 | ≤ Low cutoff | 138 | (33%) | 131 | (38%) | 7 | (9%) | <0.001 |
| | | > Low and ≤ High | 246 | (59%) | 201 | (59%) | 45 | (60%) | |
| | | > High cutoff | 33 | (8%) | 10 | (3%) | 23 | (31%) | |
| 1.3 | 9 | ≤ Low cutoff | 196 | (47%) | 186 | (54%) | 10 | (13%) | <0.001 |
| | | > Low and ≤ High | 164 | (39%) | 130 | (38%) | 34 | (45%) | |
| | | > High cutoff | 57 | (14%) | 26 | (8%) | 31 | (41%) | |
| 1.3 | 13 | ≤ Low cutoff | 196 | (47%) | 186 | (54%) | 10 | (13%) | <0.001 |
| | | > Low and ≤ High | 180 | (43%) | 143 | (42%) | 37 | (49%) | |
| | | > High cutoff | 41 | (10%) | 13 | (4%) | 28 | (37%) | |
| 1.3 | 17 | ≤ Low cutoff | 196 | (47%) | 186 | (54%) | 10 | (13%) | <0.001 |
| | | > Low and ≤ High | 188 | (45%) | 146 | (43%) | 42 | (56%) | |
| | | > High cutoff | 33 | (8%) | 10 | (3%) | 23 | (31%) | |
| 1.8 | 9 | ≤ Low cutoff | 233 | (56%) | 221 | (65%) | 12 | (16%) | <0.001 |
| | | > Low and ≤ High | 127 | (30%) | 95 | (28%) | 32 | (43%) | |
| | | > High cutoff | 57 | (14%) | 26 | (8%) | 31 | (41%) | |
| 1.8 | 13 | ≤ Low cutoff | 233 | (56%) | 221 | (65%) | 12 | (16%) | <0.001 |
| | | > Low and ≤ High | 143 | (34%) | 108 | (32%) | 35 | (47%) | |
| | | > High cutoff | 41 | (10%) | 13 | (4%) | 28 | (37%) | |
| 1.8 | 17 | ≤ Low cutoff | 233 | (56%) | 221 | (65%) | 12 | (16%) | <0.001 |
| | | > Low and ≤ High | 151 | (36%) | 111 | (32%) | 40 | (53%) | |
| | | > High cutoff | 33 | (8%) | 10 | (3%) | 23 | (31%) | |

2^{nd} Sample

| NA | NA | Median of all | 1.05 | | 0.82 | | 5.97 | | <0.001 |
|---|---|---|---|---|---|---|---|---|---|
| | | results (IQR) | (0.47-3.94) | | (0.40-2.30) | | (3.10-21.38) | | |
| 0.8 | 9 | ≤ Low cutoff | 172 | (41%) | 166 | (49%) | 6 | (8%) | <0.001 |
| | | > Low and ≤ High | 190 | (46%) | 154 | (45%) | 36 | (48%) | |
| | | > High cutoff | 55 | (13%) | 22 | (6%) | 33 | (44%) | |
| 0.8 | 13 | ≤ Low cutoff | 172 | (41%) | 166 | (49%) | 6 | (8%) | <0.001 |
| | | > Low and ≤ High | 204 | (49%) | 161 | (47%) | 43 | (57%) | |
| | | > High cutoff | 41 | (10%) | 15 | (4%) | 26 | (35%) | |
| 0.8 | 17 | ≤ Low cutoff | 172 | (41%) | 166 | (49%) | 6 | (8%) | <0.001 |
| | | > Low and ≤ High | 210 | (50%) | 164 | (48%) | 46 | (61%) | |
| | | > High cutoff | 35 | (8%) | 12 | (4%) | 23 | (31%) | |
| 1.3 | 9 | ≤ Low cutoff | 228 | (55%) | 221 | (65%) | 7 | (9%) | <0.001 |
| | | > Low and ≤ High | 134 | (32%) | 99 | (29%) | 35 | (47%) | |
| | | > High cutoff | 55 | (13%) | 22 | (6%) | 33 | (44%) | |
| 1.3 | 13 | ≤ Low cutoff | 228 | (55%) | 221 | (65%) | 7 | (9%) | <0.001 |
| | | > Low and ≤ High | 148 | (35%) | 106 | (31%) | 42 | (56%) | |
| | | > High cutoff | 41 | (10%) | 15 | (4%) | 26 | (35%) | |
| 1.3 | 17 | ≤ Low cutoff | 228 | (55%) | 221 | (65%) | 7 | (9%) | <0.001 |
| | | > Low and ≤ High | 154 | (37%) | 109 | (32%) | 45 | (60%) | |
| | | > High cutoff | 35 | (8%) | 12 | (4%) | 23 | (31%) | |
| 1.8 | 9 | ≤ Low cutoff | 247 | (59%) | 239 | (70%) | 8 | (11%) | <0.001 |
| | | > Low and ≤ High | 115 | (28%) | 81 | (24%) | 34 | (45%) | |
| | | > High cutoff | 55 | (13%) | 22 | (6%) | 33 | (44%) | |
| 1.8 | 13 | ≤ Low cutoff | 247 | (59%) | 239 | (70%) | 8 | (11%) | <0.001 |
| | | > Low and ≤ High | 129 | (31%) | 88 | (26%) | 41 | (55%) | |
| | | > High cutoff | 41 | (10%) | 15 | (4%) | 26 | (35%) | |
| 1.8 | 17 | ≤ Low cutoff | 247 | (59%) | 239 | (70%) | 8 | (11%) | <0.001 |
| | | > Low and ≤ High | 135 | (32%) | 91 | (27%) | 44 | (59%) | |
| | | > High cutoff | 35 | (8%) | 12 | (4%) | 23 | (31%) | |

3^{rd} Sample

| NA | NA | Median of all | 0.99 | | 0.78 | | 7.85 | | <0.001 |
|---|---|---|---|---|---|---|---|---|---|
| | | results (IQR) | (0.44-3.12) | | (0.38-1.82) | | (2.31-19.64) | | |
| 0.8 | 9 | ≤ Low cutoff | 181 | (43%) | 175 | (51%) | 6 | (8%) | <0.001 |
| | | > Low and ≤ High | 184 | (44%) | 149 | (44%) | 35 | (47%) | |
| | | > High cutoff | 52 | (12%) | 18 | (5%) | 34 | (45%) | |
| 0.8 | 13 | ≤ Low cutoff | 181 | (43%) | 175 | (51%) | 6 | (8%) | <0.001 |
| | | > Low and ≤ High | 193 | (46%) | 153 | (45%) | 40 | (53%) | |
| | | > High cutoff | 43 | (10%) | 14 | (4%) | 29 | (39%) | |
| 0.8 | 17 | ≤ Low cutoff | 181 | (43%) | 175 | (51%) | 6 | (8%) | <0.001 |
| | | > Low and ≤ High | 202 | (48%) | 157 | (46%) | 45 | (60%) | |
| | | > High cutoff | 34 | (8%) | 10 | (3%) | 24 | (32%) | |
| 1.3 | 9 | ≤ Low cutoff | 231 | (55%) | 221 | (65%) | 10 | (13%) | <0.001 |
| | | > Low and ≤ High | 134 | (32%) | 103 | (30%) | 31 | (41%) | |
| | | > High cutoff | 52 | (12%) | 18 | (5%) | 34 | (45%) | |
| 1.3 | 13 | ≤ Low cutoff | 231 | (55%) | 221 | (65%) | 10 | (13%) | <0.001 |
| | | > Low and ≤ High | 143 | (34%) | 107 | (31%) | 36 | (48%) | |
| | | > High cutoff | 43 | (10%) | 14 | (4%) | 29 | (39%) | |
| 1.3 | 17 | ≤ Low cutoff | 231 | (55%) | 221 | (65%) | 10 | (13%) | <0.001 |
| | | > Low and ≤ High | 152 | (36%) | 111 | (32%) | 41 | (55%) | |
| | | > High cutoff | 34 | (8%) | 10 | (3%) | 24 | (32%) | |
| 1.8 | 9 | ≤ Low cutoff | 268 | (64%) | 255 | (75%) | 13 | (17%) | <0.001 |
| | | > Low and ≤ High | 97 | (23%) | 69 | (20%) | 28 | (37%) | |
| | | > High cutoff | 52 | (12%) | 18 | (5%) | 34 | (45%) | |
| 1.8 | 13 | ≤ Low cutoff | 268 | (64%) | 255 | (75%) | 13 | (17%) | <0.001 |
| | | > Low and ≤ High | 106 | (25%) | 73 | (21%) | 33 | (44%) | |
| | | > High cutoff | 43 | (10%) | 14 | (4%) | 29 | (39%) | |
| 1.8 | 17 | ≤ Low cutoff | 268 | (64%) | 255 | (75%) | 13 | (17%) | <0.001 |
| | | > Low and ≤ High | 115 | (28%) | 77 | (23%) | 38 | (51%) | |
| | | > High cutoff | 34 | (8%) | 10 | (3%) | 24 | (32%) | |

TABLE 18
Multivariable logistic regression model for PS-AKI Endpoint with initial CCL14 category
and change in CCL14 category over the first day as predictor variables - primary analysis
cohort. CCL14 categorised by 9 different combinations of high and low cutoffs.

| | | Initial Category: M | | | | | | Change in | |
|---|---|---|---|---|---|---|---|---|---|
| Low | High | Odds | | Initial Category: H | | Change in Category: Decrease | | Category: Increase | |
| Cutoff | Cutoff | ratio | | Odds ratio | | Odds ratio | p- | Odds ratio | |
| (ng/mL) | (ng/mL) | (95% CI) | p-value | (95% CI) | p-value | (95% CI) | value | (95% CI) | p-value |
| 0.8 | 9 | 7.21 | <0.001 | 61.8 | <0.001 | 0.172 | <0.001 | 3.25 | 0.006 |
| | | (3.15-19) | | (22.8-191) | | (0.070-0.377) | | (1.39-7.52) | |
| 1.3 | 9 | 10 | <0.001 | 61.5 | <0.001 | 0.179 | <0.001 | 3.85 | 0.001 |
| | | (4.69-23.5) | | (24.7-168) | | (0.076-0.385) | | (1.69-8.86) | |
| 1.8 | 9 | 12.4 | <0.001 | 55.7 | <0.001 | 0.227 | <0.001 | 4.19 | <0.001 |
| | | (5.84-28) | | (23.5-143) | | (0.101-0.48) | | (1.85-9.62) | |
| 0.8 | 13 | 7.17 | <0.001 | 105 | <0.001 | 0.193 | <0.001 | 3.19 | 0.008 |
| | | (3.13-19) | | (35.3-357) | | (0.075-0.437) | | (1.33-7.57) | |
| 1.3 | 13 | 9.91 | <0.001 | 107 | <0.001 | 0.201 | <0.001 | 4.04 | 0.001 |
| | | (4.64-23.3) | | (39-327) | | (0.082-0.446) | | (1.75-9.46) | |
| 1.8 | 13 | 11.9 | <0.001 | 97 | <0.001 | 0.262 | 0.001 | 4.6 | <0.001 |
| | | (5.65-26.9) | | (37.1-278) | | (0.114-0.57) | | (2.01-10.7) | |
| 0.8 | 17 | 8.05 | <0.001 | 96.3 | <0.001 | 0.142 | <0.001 | 2.64 | 0.050 |
| | | (3.5-21.6) | | (30.8-348) | | (0.049-0.342) | | (0.972-6.97) | |
| 1.3 | 17 | 11.7 | <0.001 | 101 | <0.001 | 0.148 | <0.001 | 3.51 | 0.007 |
| | | (5.49-27.8) | | (34.9-330) | | (0.055-0.342) | | (1.4-8.88) | |
| 1.8 | 17 | 15.2 | <0.001 | 97.1 | <0.001 | 0.19 | <0.001 | 4.26 | 0.002 |
| | | (7.21-34.9) | | (35-300) | | (0.078-0.422) | | (1.74-10.7) | |

TABLE 19
Risk of PS-AKI stratified by initial CCL14 category and subsequent change in CCL14
category. CCL14 categorised by 9 different combinations of high and low cutoffs.

| | | Initial Category: L | | Initial Category: M | | | Initial Category: H | |
|---|---|---|---|---|---|---|---|---|
| | | Change | Change | Change | Change | Change | Change | Change |
| Low | High | from | from | from | from | from | from | from |
| Cutoff | Cutoff | initial: | initial: | initial: | initial: | initial: | initial: | initial: |
| (ng/ml) | (ng/ml) | Stable | Increase | Decrease | Stable | Increase | Decrease | Stable |
| 0.8 | 9 | 4% | 9% | 4% | 21% | 47% | 30% | 68% |
| 1.3 | 9 | 3% | 16% | 5% | 29% | 50% | 30% | 68% |
| 1.8 | 9 | 2% | 20% | 10% | 36% | 50% | 30% | 68% |
| 0.8 | 13 | 4% | 9% | 4% | 21% | 47% | 50% | 76% |
| 1.3 | 13 | 3% | 16% | 5% | 28% | 50% | 50% | 76% |
| 1.8 | 13 | 2% | 20% | 9% | 34% | 50% | 50% | 76% |
| 0.8 | 17 | 4% | 9% | 4% | 24% | 45% | 43% | 77% |
| 1.3 | 17 | 3% | 16% | 5% | 32% | 45% | 43% | 77% |
| 1.8 | 17 | 2% | 20% | 9% | 39% | 45% | 43% | 77% |

While the invention has been described and exemplified in sufficient detail for those skilled in this art to make and use it, various alternatives, modifications, and improvements should be apparent without departing from the spirit and scope of the invention. The examples provided herein are representative of preferred aspects, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

Other aspects are set forth within the following claims.

What is claimed is:

1. A method for assessing an increasing risk for developing persistent acute kidney injury (AKI) in a subject, the method comprising:
(a) performing an assay to detect a level of C—C motif chemokine 14 (CCL14) in two or more urine samples obtained from the subject, wherein at least a second urine sample is obtained from the subject within about 72 hours after a first urine sample is obtained; and
(b) determining the subject has the increasing risk of persistent AKI based upon the levels of CCL14 detected in the urine samples trending upward.

2. The method of claim 1, further comprising:
(a) performing an assay to detect a level of C—C motif chemokine 14 (CCL14) in two or more urine samples obtained from the subject, wherein at least a second urine sample is obtained from the subject within about 72 hours after a first urine sample is obtained;
(b) determining the level of CCL14 detected in the urine sample is low, medium, or high based on a first predetermined threshold concentration of CCL14 and a second predetermined threshold concentration of CCL14, wherein,
(i) when the level of C—C motif chemokine 14 in the urine sample is below the first predetermined threshold concentration of CCL14, the level of CCL14 in the urine sample is low;
(ii) when the level of CCL14 in the urine sample is above the second predetermined threshold concentration of CCL14, the level of CCL14 in the urine sample is high; or
(iii) when the level of CCL14 in the urine sample is between the first predetermined threshold concentration of CCL14 and the second predetermined threshold concentration of CCL14, the level of CCL14 in the urine sample is medium; and
(c) correlating the level of CCL14 to the increasing risk that the subject will develop persistent KDIGO stage 1, 2, or 3 AKI when
(iv) the level of CCL14 in the first urine sample is low and the level of CCL14 in the second urine sample is medium or high; or
(v) the level of CCL14 in the first urine sample is medium and the level of CCL14 in the second urine sample is high.

3. The method of claim 1 or 2, wherein the subject has AKI meeting the definition of KDIGO stage 1.

4. The method of claim 1 or 2, wherein the subject has AKI meeting the definition of KDIGO stage 2.

5. The method of claim 1 or 2, wherein the subject has AKI meeting the definition of KDIGO stage 3.

6. The method of any one of claims 1-5, wherein the subject is determined to have the increasing risk of developing the persistent KDIGO stage 2 or 3 AKI.

7. The method of any one of claims 1-6, wherein the subject is determined to have the increasing risk of developing the persistent KDIGO stage 3 AKI.

8. The method of any one of claims 1-7, comprising determining that the subject is at an increasing risk of developing a persistent KDIGO stage 3 AKI within 48 hours of the time the first urine sample is obtained, wherein the duration of the persistent KDIGO stage 3 AKI comprises a 72-hour period with a minimum KDIGO stage of KDIGO 3.

9. The method of any one of claims 2-8, wherein the first threshold concentration of CCL14 is less than or equal to 1.3 ng/ml of CCL14.

10. The method of any one of claims 2-9, wherein the second threshold concentration of CCL14 is greater than 13 ng/mL.

11. The method of any one of claims 1-10, wherein the second urine sample is obtained within about 12 hours of the first urine sample.

12. The method of any one of claims 1-10, wherein the second urine sample is obtained about 12 hours after the first urine sample.

13. The method of any one of claims 1-10, wherein the second urine sample is obtained within about 24 hours of the first urine sample.

14. The method of any one of claims 1-10, wherein the second urine sample is obtained about 24 hours after the first urine sample.

15. The method of any one of claims 1-10, wherein the second urine sample is obtained within about 36 hours of the first urine sample.

16. The method of any one of claims 1-10, wherein the second urine sample is obtained about 36 hours after the first urine sample.

17. The method of any one of claims 1-10, wherein the second urine sample is obtained within about 48 hours of the first urine sample.

18. The method of any one of claims 1-10, wherein the second urine sample is obtained about 48 hours after the first urine sample.

19. The method of any one of claims 1-10, wherein the second urine sample is obtained within about 72 hours of the first urine sample.

20. The method of any one of claims 1-10, wherein the second urine sample is obtained about 72 hours after the first urine sample.

21. The method of any one of claims 1-20, further comprising obtaining a third urine sample.

22. The method of any one of claims 1-12, further comprising obtaining a third urine sample, wherein the second and third urine samples are obtained within about 12 hours of the first urine sample.

23. The method of any one of claims 1-14, further comprising obtaining a third urine sample, wherein the second and third urine samples are obtained within about 24 hours of the first urine sample.

24. The method of any one of claims 1-21, wherein the second urine sample is obtained at about 12 hours after the first urine sample is obtained and further comprising obtaining a third urine sample at about 24 hours after the first urine sample is obtained.

25. The method of any one of claims 21-24, wherein the first urine sample and the second urine sample have a low level of CCL14 and the third urine sample has a medium level of CCL14.

26. The method of any one of claims 21-24, wherein the first urine sample and the second urine sample have a low level of CCL14 and the third urine sample has a high level of CCL14.

27. The method of any one of claims 21-24, wherein the first urine sample and the second urine sample have a medium level of CCL14 and the third urine sample has a high level of CCL14.

28. The method of any one of claims 21-24, wherein the first urine sample has a low level of CCL14 and the second urine sample and the third urine sample have a medium level of CCL14.

29. The method of any one of claims 21-24, wherein the first urine sample has a medium level of CCL14 and the second urine sample and the third urine sample have a high level of CCL14.

30. The method of any one of claims 21-24, wherein the first urine sample has a low level of CCL14, the second urine sample has a medium level of CCL14, and the third sample has a high level of CCL14.

31. The method of any one of claims 21-24, wherein the first urine sample has a low level of CCL14, and the second urine sample and the third urine sample have a high level of CCL14.

32. The method of any one of claims 1-31, wherein the subject is in the intensive care unit.

33. The method of any one of claims 1-32, wherein the subject is diagnosed as having AKI.

34. The method of claim 33, wherein the subject has had AKI for less than 36 hours before the first urine sample is obtained.

35. The method of any one of claims 1-34, wherein the method further comprises contacting the urine sample with a binding reagent which binds to the CCL14.

36. The method of claim 35, wherein the binding reagent is an antibody.

37. The method of any one of claims 1-36, wherein the assay is an immunoassay.

38. The method of any one of claims 1-37, further comprising treating the subject at an increasing risk of having persistent AKI with one or more of renal replacement therapy (RRT), withdrawing of compounds that are known to be damaging to the kidney, performing a procedure known to be damaging to the kidney after a delay of at least about 48 hours from obtaining the first or second sample, modifying diuretic administration, modifying dosing of renally cleared compounds, and/or administering one or more agents or measured volumes of fluid to restore normal fluid levels, electrolyte levels, or hemodynamics.

39. The method of claim 38, wherein the RRT comprises one or more of continuous RRT, intermittent RRT, hemodialysis, peritoneal dialysis, hemofiltration, and renal transplantation.

40. A method for assessing a decreasing risk for developing persistent acute kidney injury (AKI) in a subject, the method comprising:
(a) performing an assay to detect a level of C—C motif chemokine 14 (CCL14) in two or more urine samples obtained from the subject, wherein at least a second urine sample is obtained from the subject within about 72 hours after a first urine sample is obtained; and
(b) determining the subject is at decreasing risk of persistent AKI based upon the levels of CCL14 detected in the urine samples trending downward.

41. The method of claim 40, further comprising:
(a) performing an assay to detect a level of C—C motif chemokine 14 (CCL14) in two or more urine samples obtained from the subject, wherein at least a second urine sample is obtained from the subject within about 72 hours after a first urine sample is obtained;
(b) determining the level of CCL14 detected in the urine sample is low, medium, or high based on a first predetermined threshold concentration of CCL14 and a second predetermined threshold concentration of CCL14, wherein,
(i) when the level of C—C motif chemokine 14 in the urine sample is below the first predetermined threshold concentration of CCL14, the level of CCL14 in the urine sample is low;
(ii) when the level of CCL14 in the urine sample is above the second predetermined threshold concentration of CCL14, the level of CCL14 in the urine sample is high; or
(iii) when the level of CCL14 in the urine sample is between the first predetermined threshold concentration of CCL14 and the second predetermined threshold concentration of CCL14, the level of CCL14 in the urine sample is medium; and
(c) correlating the level of CCL14 to the decreasing risk that the subject will develop persistent KDIGO stage 1, 2, or 3 AKI when (iv) the level of CCL14 in the first urine sample is medium and the level of CCL14 in the second urine sample is low;
(v) the level of CCL14 in the first urine sample is high and the level of CCL14 in the second urine sample is medium; or
(vi) the level of CCL14 in the first urine sample is high and the level of CCL14 in the second urine sample is low.

42. The method of claim 40 or 41, wherein the subject has AKI meeting the definition of KDIGO stage 1.

43. The method of claim 40 or 41, wherein the subject has AKI meeting the definition of KDIGO stage 2.

44. The method of claim 40 or 41, wherein the subject has AKI meeting the definition of KDIGO stage 3.

45. The method of any one of claims-40-44, wherein the subject is determined to be at decreasing risk of developing the persistent KDIGO stage 2 or 3 AKI.

46. The method of any one of claims 40-45 wherein the subject is determined to be at decreasing risk of developing the persistent KDIGO stage 3 AKI.

47. The method of any one of claims 40-46, comprising determining that the subject is at a decreasing risk of developing a persistent KDIGO stage 3 AKI within 48 hours of the time the first urine sample is obtained, wherein the duration of the persistent KDIGO stage 3 AKI comprises a 72-hour period with a minimum KDIGO stage of KDIGO 3.

48. The method of any one of claims 41-47, wherein the first threshold concentration of CCL14 is less than or equal to 1.3 ng/ml of CCL14.

49. The method of any one of claims 41-48, wherein the second threshold concentration of CCL14 is greater than 13 ng/mL.

50. The method of any one of claims 40-49, wherein the second urine sample is obtained within about 12 hours of the first urine sample.

51. The method of any one of claims 40-49, wherein the second urine sample is obtained about 12 hours after the first urine sample.

52. The method of any one of claims 40-49, wherein the second urine sample is obtained within about 24 hours of the first urine sample.

53. The method of any one of claims 40-49, wherein the second urine sample is obtained about 24 hours after the first urine sample.

54. The method of any one of claims 40-49, wherein the second urine sample is obtained within about 36 hours of the first urine sample.

55. The method of any one of claims 40-49, wherein the second urine sample is obtained about 36 hours after the first urine sample.

56. The method of any one of claims 40-49, wherein the second urine sample is obtained within about 48 hours of the first urine sample.

57. The method of any one of claims 40-49, wherein the second urine sample is obtained about 48 hours after the first urine sample.

58. The method of any one of claims 40-49, wherein the second urine sample is obtained within about 72 hours of the first urine sample.

59. The method of any one of claims 40-49, wherein the second urine sample is obtained about 72 hours after the first urine sample.

60. The method of any one of claims 40-59, further comprising obtaining a third urine sample.

61. The method of any one of claims 40-51, further comprising obtaining a third urine sample, wherein the second and third urine samples are obtained within about 12 hours of the first urine sample.

62. The method of any one of claims 40-53, further comprising obtaining a third urine sample, wherein the second and third urine samples are obtained within about 24 hours of the first urine sample.

63. The method of any one of claim 40-53 or 60, wherein the second urine sample is obtained at about 12 hours after the first urine sample is obtained and further comprising obtaining a third urine sample at about 24 hours after the first urine sample is obtained.

64. The method of any one of claims 60-63, wherein the first urine sample and the second urine sample have a high level of CCL14 and the third urine sample has a medium level of CCL14.

65. The method of any one of claims 60-63, wherein the first urine sample and the second urine sample have a high level of CCL14 and the third urine sample has a low level of CCL14.

66. The method of any one of claims 60-63, wherein the first urine sample and the second urine sample have a medium level of CCL14 and the third urine sample has a low level of CCL14.

67. The method of any one of claims 60-63, wherein the first urine sample has a medium level of CCL14 and the second urine sample and the third urine sample have a low level of CCL14.

68. The method of any one of claims 60-63, wherein the first urine sample has a high level of CCL14 and the second urine sample and the third urine sample have a low level of CCL14.

69. The method of any one of claims 60-63, wherein the first urine sample has a high level of CCL14 and the second urine sample and the third urine sample have a medium level of CCL14.

70. The method of any one of claims 60-63, wherein the first urine sample has a high level of CCL14, the second urine sample has a medium level of CCL14, and the third urine sample has a low level of CCL14.

71. The method of any one of claims 40-70, wherein the subject is in the intensive care unit.

72. The method of any one of claims 40-71, wherein the subject is diagnosed as having AKI.

73. The method of claim 72, wherein the subject has had AKI for less than 36 hours before the first urine sample is obtained.

74. The method of any one of claims 40-73, wherein the method further comprises contacting the urine sample with a binding reagent which binds to the CCL14.

75. The method of claim 74, wherein the binding reagent is an antibody.

76. The method of any one of claims 40-75, wherein the assay is an immunoassay.

77. The method of any one of claims 40-76, further comprising treating the subject at decreasing risk of persistent AKI by one or more of administering compounds that are known to be damaging to the kidney, performing a procedure known to be damaging to the kidney, modifying diuretic administration, modifying dosing of renally cleared compounds, and administering one or more agents or measured volumes of fluid to restore normal fluid levels, electrolyte levels, or hemodynamics.

78. A method for assessing an elevated risk for developing persistent acute kidney injury (AKI) in a subject, the method comprising:
(a) performing an assay to detect a level of C—C motif chemokine ligand 14 (CCL14) in a urine sample obtained from the subject; and
(b) determining the subject is at elevated risk for developing persistent AKI based upon the level of CCL14 detected in the urine sample being greater than a CCL14 threshold concentration of about 1.3 ng/ml.

79. A method for assessing a high risk for developing persistent acute kidney injury (AKI) in a subject, the method comprising:
(a) performing an assay to detect a level of C—C motif chemokine ligand14 (CCL14) in a urine sample obtained from the subject; and
(b) determining the subject is at high risk for developing persistent AKI based upon the level of CCL14 detected in the urine sample being greater than a CCL14 threshold concentration of about 13.0 ng/ml.

80. The method of claim 78, wherein the level of CCL14 detected in the urine sample is above the CCL14 threshold concentration of about 1.3 ng/ml and below a CCL14 threshold concentration of about 13.0 ng/ml.

81. The method of any one of claims 78-80, wherein the method further comprises seeking further analysis from a nephrologist or specialist.

82. The method of any one of claim 78-81, further comprising treating the subject at elevated or high risk of having persistent AKI with one or more of renal replacement therapy (RRT), withdrawing of compounds that are known to be damaging to the kidney, performing a procedure known to be damaging to the kidney after a delay of at least about 48 hours from obtaining the sample, modifying diuretic administration, modifying dosing of renally cleared compounds, and/or administering one or more agents or measured volumes of fluid to restore normal fluid levels, electrolyte levels, or hemodynamics.

83. The method of claim 79, wherein the RRT comprises one or more of continuous RRT, intermittent RRT, hemodialysis, peritoneal dialysis, hemofiltration, and renal transplantation.

84. The method of any one of claims 78-83, wherein the subject is diagnosed as having AKI.

85. The method of any one of claims 78-83, wherein the subject has KDIGO stage 1 AKI.

86. The method of any one of claims 78-83, wherein the subject has KDIGO stage 2 AKI.

87. The method of any one of claims 78-83, wherein the subject has KDIGO stage 3 AKI.

88. The method of any one of claims 78-87, wherein the subject is determined to be at elevated or high risk of persistent KDIGO stage 2 or 3 AKI.

89. The method of any one of claims 78-89, wherein the subject is determined to be at elevated or high risk of persistent KDIGO stage 3 AKI.

90. The method of any one of claims 78-89, comprising determining that the subject is at an elevated or high risk of developing a persistent KDIGO stage 3 AKI within 48 hours of the time at which the urine sample is obtained, wherein the duration of the persistent KDIGO stage 3 AKI comprises a 72-hour period with a minimum KDIGO stage of KDIGO 3.

91. The method of any one of claims 78-90, wherein the subject is in an intensive care unit.

92. The method of any one of claims 78-91, wherein the method further comprises contacting the urine sample with a binding reagent that binds to the CCL14.

93. The method of claim 92 wherein the binding reagent is an antibody.

94. The method of any one of claims 78-93, wherein the assay is an immunoassay.

95. A method for assessing an elevated risk for developing persistent acute kidney injury (AKI) in a subject, the method comprising:
(a) performing an assay to detect a level of C—C motif chemokine ligand 14 (CCL14) in a urine sample obtained from the subject; and
(b) correlating the assay result to an elevated risk for the subject developing persistent AKI by comparing the assay result to a CCL14 threshold concentration of about 1.3 ng/ml;
wherein the correlation is used as a rule-in test for the elevated risk for the subject developing persistent AKI when the assay result is above the CCL14 threshold concentration of about 1.3 ng/ml, or
wherein the correlation is used as a rule-out test for the subject having the elevated risk of developing persistent AKI when the assay result is below the CCL14 threshold concentration of about 1.3 ng/ml.

96. A method for assessing a high risk for developing persistent acute kidney injury (AKI) in a subject, the method comprising:
(a) performing an assay to detect a level of C—C motif chemokine ligand 14 (CCL14) in a urine sample obtained from the subject; and
(b) correlating the assay result to a high risk for the subject developing persistent AKI by comparing the assay result to a CCL14 threshold concentration of about 13.0 ng/ml;
wherein the correlation is used as a rule-in test for the subject having the high risk of developing persistent AKI when the assay result is above the CCL14 threshold concentration of about 13.0 ng/ml, or
wherein the correlation is used as a rule-out test for the subject having the high risk of developing persistent AKI when the assay result is below the CCL14 threshold concentration of about 13.0 ng/ml.

97. The method of claim 95, wherein the level of CCL14 detected in the urine sample is above the CCL14 threshold concentration of about 1.3 ng/ml and below a CCL14 threshold concentration of about 13.0 ng/ml.

98. The method of any one of claims 95-97, wherein the method further comprises seeking further analysis from a nephrologist or specialist.

99. The method of any one of claims 95-97, further comprising treating the subject at elevated or high risk of having persistent AKI with one or more of renal replacement therapy (RRT), withdrawing of compounds that are known to be damaging to the kidney, performing a procedure known to be damaging to the kidney after a delay of at least about 48 hours from obtaining the sample, modifying diuretic administration, modifying dosing of renally cleared compounds, and/or administering one or more agents or measured volumes of fluid to restore normal fluid levels, electrolyte levels, or hemodynamics.

100. The method of claim 99, wherein the RRT comprises one or more of continuous RRT, intermittent RRT, hemodialysis, peritoneal dialysis, hemofiltration, and renal transplantation.

101. The method of any one of claims 95-100, wherein the subject is diagnosed as having AKI.

102. The method of any one of claims 95-101, wherein the subject has KDIGO stage 1 AKI.

103. The method of any one of claims 95-101, wherein the subject has KDIGO stage 2 AKI.

104. The method of any one of claims 95-101, wherein the subject has KDIGO stage 3 AKI.

105. The method of any one of claims 95-104, wherein the subject is determined to have an elevated or high risk of persistent KDIGO stage 2 or 3 AKI.

106. The method of any one of claims 92-105, wherein the subject is determined to have an elevated or high risk of persistent KDIGO stage 3 AKI.

107. The method of any one of claims 92-106, comprising determining that the subject is at an elevated or high risk of developing persistent KDIGO stage 3 AKI within 48 hours of the time at which the urine sample is obtained, wherein the duration of the persistent KDIGO stage 3 AKI comprises a 72-hour period with a minimum KDIGO stage of KDIGO 3.

108. The method of any one of claims 95-107, wherein the subject is in an intensive care unit.

109. The method of any one of claims 95-108, wherein the method further comprises contacting the urine sample with a binding reagent that binds to the CCL14.

101. The method of claim 109, wherein the binding reagent is an antibody.

111. The method of any one of claims 95-110, wherein the assay is an immunoassay.

112. A method for assessing a low risk for developing persistent acute kidney injury (AKI) in a subject, the method comprising:
(a) performing an assay to detect a level of C—C motif chemokine ligand 14 (CCL14) in a urine sample obtained from the subject; and
(b) determining the subject is at low risk for developing persistent AKI based upon the level of CCL14 detected in the urine sample being less than a CCL14 threshold concentration of about 1.3 ng/ml.


SEQUENCE LISTING (2 sequences)

SEQ ID NO: 1 - protein, 93 aa, Homo sapiens
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 2 - protein, 17 aa, Homo sapiens
QTGGKPKVVKIQLKLVG